(19) United States
(12) Patent Application Publication
(10) Pub. No.: US 2017/0222159 A1
(43) Pub. Date: Aug. 3, 2017

(54) ELECTRON BUFFERING MATERIAL AND ORGANIC ELECTROLUMINESCENT DEVICE

(71) Applicant: Rohm and Haas Electronic Materials Korea Ltd., Cheonan (KR)

(72) Inventors: Jeong-Eun Yang, Suwon (KR); Su-Hyun Lee, Suwon (KR); Chi-Sik Kim, Hwaseong (KR); Young-Jun Cho, Seongnam (KR); Kyung-Hoon Choi, Hwaseong (KR); Sang-Hee Cho, Suwon (KR)

(21) Appl. No.: 15/329,136

(22) PCT Filed: Jul. 29, 2015
(86) PCT No.: PCT/KR2015/007941

§ 371 Date: Jan. 25, 2017

(30) Foreign Application Priority Data
Jul. 29, 2014 (KR) 10-2014-0096462

(51) Int. Cl. H01L 51/00; C07D 495/04; C07D 491/048

(52) U.S. Cl. CPC H01L 51/0071; C07D 491/048; H01L 51/0067; C07D 495/04; H01L 51/5008

(57) ABSTRACT

The present invention relates to an electron buffering material, and an organic electroluminescent device comprising a first electrode; a second electrode facing the first electrode; a light-emitting layer between the first electrode and the second electrode; and an electron transport zone and an electron buffering layer between the light-emitting layer and the second electrode. The electron buffering material of the present invention can produce an organic electroluminescent device having low driving voltage, excellent luminous efficiency, and long lifespan.

### TECHNICAL FIELD

The present invention relates to an electron buffering material, and an organic electroluminescent device comprising the same.

### BACKGROUND ART

After Tang et al. of Eastman Kodak first developed a TPD/Alq3 bilayer small molecule green organic electroluminescent device (OLED) composed of a light-emitting layer and an charge transport layer in 1987, studies of organic electroluminescent devices have been rapidly conducted, and now became commercialized. At present, phosphorescent materials, which have excellent luminous efficiency, are mainly used for panels of the organic electroluminescent devices. In the case of red and green light-emitting organic electroluminescent devices, commercialization of organic electroluminescent devices using phosphorescent materials succeeded. However, in the case of blue phosphorescent materials, characteristics deteriorate due to decrease of roll-off at high current by loss of excessively formed excitons, the blue phosphorescent material itself has problems in long-term lifespan stability, and the color purity sharply drops as time passes, which are obstacles to the realization of a full color display.

The fluorescent materials used at present also have several problems. First, when exposed to high temperature during a process of producing a panel, a current characteristic of the device changes to cause a problem of a change in luminance, and due to a structural characteristic, a drop of an interfacial characteristic between a light-emitting layer and an electron injection layer causes a decrease in luminance. In addition, a fluorescent material provides lower efficiencies than a phosphorescent material. Accordingly, there have been attempts to improve efficiencies by developing a specific fluorescent material such as a combination of an anthracene-based host and a pyrene-based dopant. However, the proposed combination makes holes become greatly trapped, which can cause light-emitting sites in a light-emitting layer to shift to the side close to a hole transport layer, thereby light being emitted at an interface. The light-emission at the interface decreases lifespan of a device, and efficiencies are not satisfactory.

It is not easy to solve the aforementioned problems of a fluorescent material by improving a light-emitting material itself. Accordingly, recently, there have been attempts to solve the problems, which include improvement in a charge transport material to change a charge transport feature, and development of an optimized device structure.

Korean Patent Application Laying-Open No. 10-2012-0092550 discloses an organic electroluminescent device in which a blocking layer is interposed between an electron injection layer and a light-emitting layer, wherein the blocking layer comprises an aromatic heterocyclic derivative comprising an azine ring. However, the prior art reference fails to disclose an organic electroluminescent device using a compound in which a benzofuran or benzothiophene is fused to a carbazole derivative to form a backbone of the compound, in an electron buffering layer.

Japanese Patent No. 4947909 discloses a blue fluorescent light-emitting device comprising an electron buffering layer wherein electrons are efficiently injected to the light-emitting layer compared to Alq3 by inserting the electron buffering layer, and the mobility of the electrons is controlled to lower the driving voltage of the device and enhance lifespan by preventing degradation of the light-emitting interface. However, the electron buffering materials are limited to Alq3 derivatives, and have limited objectives to block electrons, and the group of compounds disclosed as buffering materials is small. Thus, they have limitations in analyzing materials having improved luminous efficiency and lifespan.

### DISCLOSURE OF THE INVENTION

### Problems to be Solved

The objective of the present invention is to provide an electron buffering material which can produce an organic electroluminescent device having low driving voltage and excellent luminous efficiency, and an organic electroluminescent device comprising the same.

### Solution to Problems

The present inventors have found that the objective above can be achieved by an electron buffering material comprising a compound represented by the following formula 1, and an organic electroluminescent device comprising a first electrode; a second electrode facing the first electrode; a light-emitting layer between the first electrode and the second electrode; and an electron transport zone and an electron buffering layer between the light-emitting layer and the second electrode; wherein the electron buffering layer comprises a compound represented by the following formula 1:

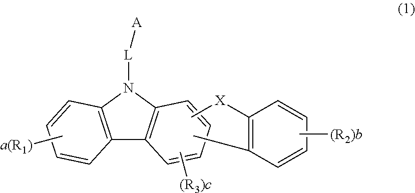

wherein

X represents O or S;

L represents a single bond, a substituted or unsubstituted (C6-C30)arylene, or a substituted or unsubstituted 5- to 30-membered heteroarylene;

A represents a substituted or unsubstituted 5- to 30-membered heteroaryl;

R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted 5- to 30-membered heteroaryl, a substituted or unsubstituted (C6-C30)aryl(C1-C30)alkyl, a substituted or unsubstituted (C3-C30)cycloalkyl, a substituted or unsubstituted (C1-C30)alkoxy, a substituted or unsubstituted (C1-C30)alkylsilyl, a substituted or unsubstituted (C6-C30)arylsilyl, a substituted or unsubstituted (C6-C30)aryl(C1-C30)alkylsilyl, a substituted or unsubstituted (C1-C30)alkylamino, a substituted or unsubstituted (C6-C30)arylamino, or a substituted or unsubstituted (C1-C30)alkyl(C6-C30)arylamino; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur;

R₃ represents hydrogen, deuterium, a halogen, a cyano, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur;

a and b each independently represent an integer of 1 to 4; where a or b is an integer of 2 or more, each of R₁ and each of R₂ may be the same or different;

c represents an integer of 1 to 2; where c is 2, each of R₃ may be the same or different; and

the heteroaryl(ene) contains at least one hetero atom selected from B, N, O, S, Si, and P.

### Effects of the Invention

By comprising the electron buffering material according to the present invention, an organic electroluminescent device can obtain a fast electron current characteristic due to a planar structure by controlling the intermolecular 7-orbital characteristics, and accordingly exhibit excellent efficiency and low driving voltage.

### BRIEF DESCRIPTION OF THE FIGURES

FIG. 1 is a schematic sectional view illustrating the structure of an organic electroluminescent device according to one embodiment of the present invention.

FIG. 2 is an energy band diagram of a hole transport layer, a light-emitting layer, an electron buffering layer, and an electron transport zone of an organic electroluminescent device according to one embodiment of the present invention.

FIG. 3 is a graph illustrating current efficiency versus luminance of the organic electroluminescent devices of Device Example 1 and Comparative Example 1.

### EMBODIMENTS OF THE INVENTION

Hereinafter, the present invention will be described in detail. However, the following description is intended to explain the invention, and is not meant in any way to restrict the scope of the invention.

Herein, “(C1-C30)alkyl” indicates a linear or branched alkyl chain having 1 to 30, preferably 1 to 10, and more preferably 1 to 6 carbon atoms constituting the chain, and includes methyl, ethyl, n-propyl, isopropyl, n-butyl, isobutyl, tert-butyl, etc. “(C2-C30) alkenyl” indicates a linear or branched alkenyl chain having 2 to 30, preferably 2 to 20, and more preferably 2 to 10 carbon atoms constituting the chain and includes vinyl, 1-propenyl, 2-propenyl, 1-butenyl, 2-butenyl, 3-butenyl, 2-methylbut-2-enyl, etc. “(C2-C30)alkynyl” indicates a linear or branched alkynyl chain having 2 to 30, preferably 2 to 20, and more preferably 2 to 10 carbon atoms constituting the chain and includes ethynyl, 1-propynyl, 2-propynyl, 1-butynyl, 2-butynyl, 3-butynyl, 1-methylpent-2-ynyl, etc. “(C3-C30)cycloalkyl” indicates a mono- or polycyclic hydrocarbon having 3 to 30, preferably 3 to 20, and more preferably 3 to 7 ring backbone carbon atoms and includes cyclopropyl, cyclobutyl, cyclopentyl, cyclohexyl, etc. “3- to 7-membered heterocycloalkyl” indicates a cycloalkyl having 3 to 7 ring backbone atoms including at least one hetero atom selected from B, N, O, S, Si, and P, preferably O, S, and N, and includes tetrahydrofuran, pyrrolidine, thiolan, tetrahydropyran, Furthermore, “(C6-C30)aryl(ene)” indicates a monocyclic or fused ring-based radical derived from an aromatic hydrocarbon and having 6 to 30, preferably 6 to 20, and more preferably 6 to 15 ring backbone carbon atoms, and includes phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, etc. “5- to 30-membered heteroaryl(ene)” indicates an aryl group having 5 to 30 ring backbone atoms including at least one, preferably 1 to 4, hetero atom selected from the group consisting of B, N, O, S, Si, and P; may be a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzonaphthothiophenyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. Furthermore, “halogen” includes F, Cl, Br, and I.

In the present invention, the compound represented by formula 1 may be represented by one of the following formulae 2 to 7:

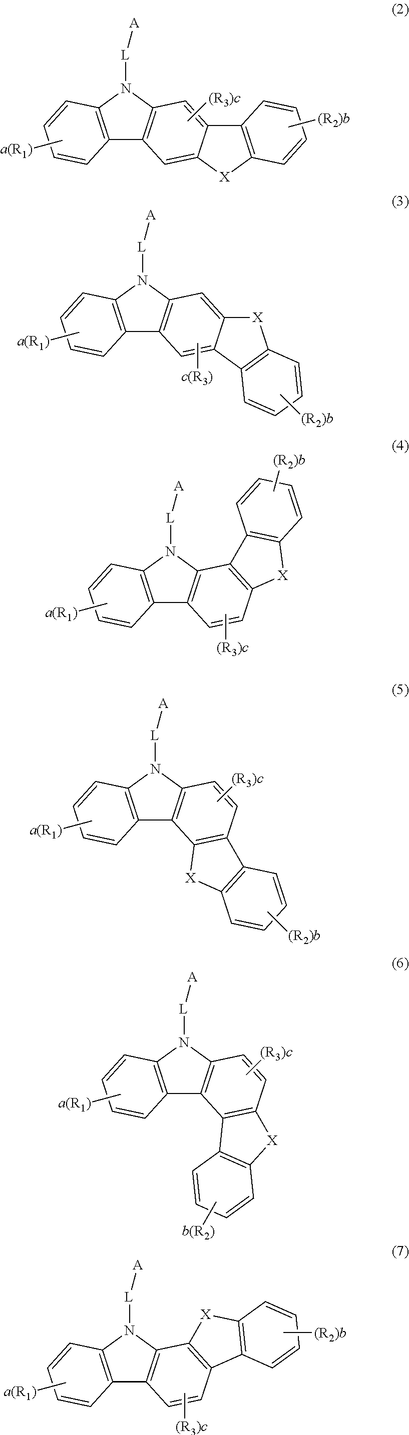

wherein X, A, L, R₁ to R₃, a, b, and c are as defined in formula 1.

Herein, “substituted” in the expression, “substituted or unsubstituted,” means that a hydrogen atom in a certain functional group is replaced with another atom or group, i.e. a substituent. In the present invention, the substituents of the substituted alkyl, the substituted alkoxy, the substituted cycloalkyl, the substituted aryl(ene), the substituted heteroaryl(ene), the substituted alkylsilyl, the substituted arylsilyl, the substituted arylalkylsilyl, the substituted arylamino, the substituted alkylamino, the substituted alkylarylamino, and the substituted arylalkyl of L, A, and R₁ to R₃ in formula 1, each independently, are at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 3- to 30-membered heteroaryl unsubstituted or substituted with a (C6-C30)aryl, a (C6-C30)aryl, a (C6-C30)aryl substituted with a 3- to 30-membered heteroaryl, a (C6-C30)aryl substituted with a tri(C1-C30)alkylsilyl, a (C6-C30)aryl substituted with a tri(C6-C30)arylsilyl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl, and preferably a cyano, a (C1-C6)alkyl, a 5- to 20-membered heteroaryl unsubstituted or substituted with a (C6-C20)aryl, a (C6-C25)aryl, a (C6-C20)aryl substituted with a 5- to 20-membered heteroaryl, a (C6-C20)aryl substituted with a tri(C1-C6)alkylsilyl, a (C6-C20)aryl substituted with a tri(C6-C20)arylsilyl, and a (C1-C6)alkyl(C6-C20)aryl.

In formula 1, X represents O or S.

L represents a single bond, a substituted or unsubstituted (C6-C30)arylene, or a substituted or unsubstituted 5- to 30-membered heteroarylene, preferably represents a single bond, a substituted or unsubstituted (C6-C20)arylene, or a substituted or unsubstituted 5- to 20-membered heteroarylene, and more preferably represents a single bond, an unsubstituted (C6-C20)arylene, or an unsubstituted 5- to 20-membered heteroarylene.

A represents a substituted or unsubstituted 5- to 30-membered heteroaryl, preferably represents a substituted or unsubstituted 5- to 25-membered heteroaryl, and more preferably represents an unsubstituted 5- to 25-membered heteroaryl, a 5- to 25-membered heteroaryl substituted with a cyano, a 5- to 25-membered heteroaryl substituted with a (C6-C25)aryl, a 5- to 25-membered heteroaryl substituted with a 5- to 20-membered heteroaryl, or a 5- to 25-membered heteroaryl substituted with a (C1-C6)alkyl(C6-C20)aryl.

In the definition of A, 5- to 30-membered heteroaryl is preferably a nitrogen-containing heteroaryl, and more preferably, is a substituted or unsubstituted pyridine, a substituted or unsubstituted pyrimidine, a substituted or unsubstituted triazine, a substituted or unsubstituted pyrazine, a substituted or unsubstituted quinoline, a substituted or unsubstituted quinazoline, a substituted or unsubstituted quinoxaline, a substituted or unsubstituted benzimidazole, a substituted or unsubstituted naphthyridine, or a substituted or unsubstituted phenanthroline.

R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted 5- to 30-membered heteroaryl, a substituted or unsubstituted (C6-C30)aryl(C1-C30)alkyl, a substituted or unsubstituted (C3-C30)cycloalkyl, a substituted or unsubstituted (C1-C30)alkoxy, a substituted or unsubstituted (C1-C30)alkylsilyl, a substituted or unsubstituted (C6-C30)arylsilyl, a substituted or unsubstituted (C6-C30)aryl(C1-C30)alkylsilyl, a substituted or unsubstituted (C1-C30)alkylamino, a substituted or unsubstituted (C6-C30)arylamino, or a substituted or unsubstituted (C1-C30)alkyl(C6-C30)arylamino; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur, preferably each independently represent hydrogen, a substituted or unsubstituted (C6-C20)aryl, or a substituted or unsubstituted 5- to 20-membered heteroaryl, and more preferably each independently represent hydrogen, a (C6-C20)aryl unsubstituted or substituted with a (C1-C6)alkyl, or a 5- to 20-membered heteroaryl unsubstituted or substituted with a (C6-C20)aryl.

R₃ represents hydrogen, deuterium, a halogen, a cyano, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur, and preferably represents hydrogen.

a and b each independently represent an integer of 1 to 4, and preferably an integer of 1 to 2; where a or b is an integer of 2 or more, each of R₁ and each of R₂ may be the same or different.

c represents an integer of 1 to 2, and preferably 1; where c is 2, each of R₃ may be the same or different.

The heteroaryl(ene) contains at least one hetero atom selected from B, N, O, S, Si, and P.

According to one embodiment of the present invention, in formula 1 above, X represents O or S; L represents a single bond, a substituted or unsubstituted (C6-C20)arylene, or a substituted or unsubstituted 5- to 20-membered heteroarylene; A represents a substituted or unsubstituted 5- to 25-membered heteroaryl; R₁ and R₂ each independently represent hydrogen, a substituted or unsubstituted (C6-C20)aryl, or a substituted or unsubstituted 5- to 20-membered heteroaryl; R₃ represents hydrogen; a and b each independently represent an integer of 1 to 2; and c represents 1.

According to another embodiment of the present invention, in formula 1 above, X represents O or S; L represents a single bond, an unsubstituted (C6-C20)arylene, or an unsubstituted 5- to 20-membered heteroarylene; A represents an unsubstituted 5- to 25-membered heteroaryl, a 5- to 25-membered heteroaryl substituted with a cyano, a 5- to 25-membered heteroaryl substituted with a (C6-C25)aryl, a 5- to 25-membered heteroaryl substituted with a 5- to 20-membered heteroaryl, or a 5- to 25-membered heteroaryl substituted with a (C1-C6)alkyl(C6-C20)aryl; R₁ and R₂ each independently represent hydrogen, a (C6-C20)aryl unsubstituted or substituted with a (C1-C6)alkyl, or a 5- to 20-membered heteroaryl unsubstituted or substituted with a (C6-C20)aryl; R₃ represents hydrogen; a and b each independently represent an integer of 1 to 2; and c represents 1.

Basically, LUMO (lowest unoccupied molecular orbital) energy and HOMO (highest occupied molecular orbital) energy levels have negative values. However, for convenience, LUMO energy level and HOMO energy level are expressed in absolute values in the present invention. In addition, the values of the LUMO energy levels are compared based on absolute values.

In the present invention, HOMO and LUMO energy levels are determined by density functional theory (DFT) calculations. The results according to the relationship between the LUMO energy level of an electron buffering layer (Ab) and the LUMO energy level of a host (Ah) are intended to explain the general tendency of a device in accordance with the overall LUMO energy groups of the electron buffering layer, and the results may be changed depending on the inherent property of specific derivatives and the stability of materials.

According to one aspect of the present invention, an electron buffering material comprising the compound represented by formula 1 is provided. The electron buffering material indicates a material controlling an electron flow. Therefore, the electron buffering material may be, for example, a material which traps electrons, blocks electrons, or lowers an energy barrier between an electron transport zone and a light-emitting layer. Specifically, the electron buffering material may be for an organic electroluminescent device. In the organic electroluminescent device, the electron buffering material may be used for preparing an electron buffering layer, or may be incorporated to another area such as an electron transport zone or a light-emitting layer. The electron buffering layer may be formed between a light-emitting layer and an electron transport zone, or between an electron transport zone and a second electrode of an organic electroluminescent device. The electron buffering material may be a mixture or composition which may further comprise materials which are conventionally used for preparing an organic electroluminescent device.

The specific compounds represented by formula 1 include the following compounds, but are not limited thereto:

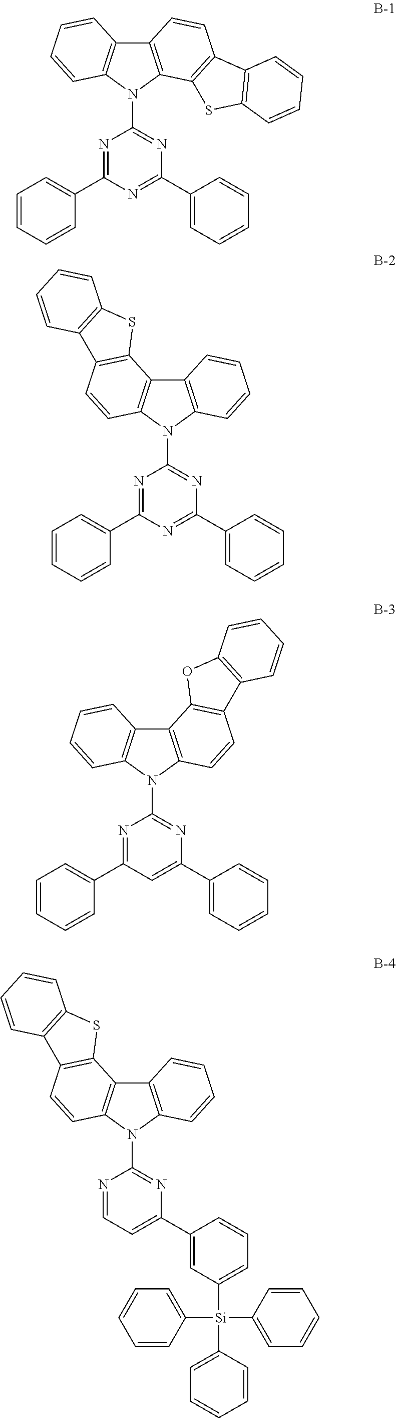

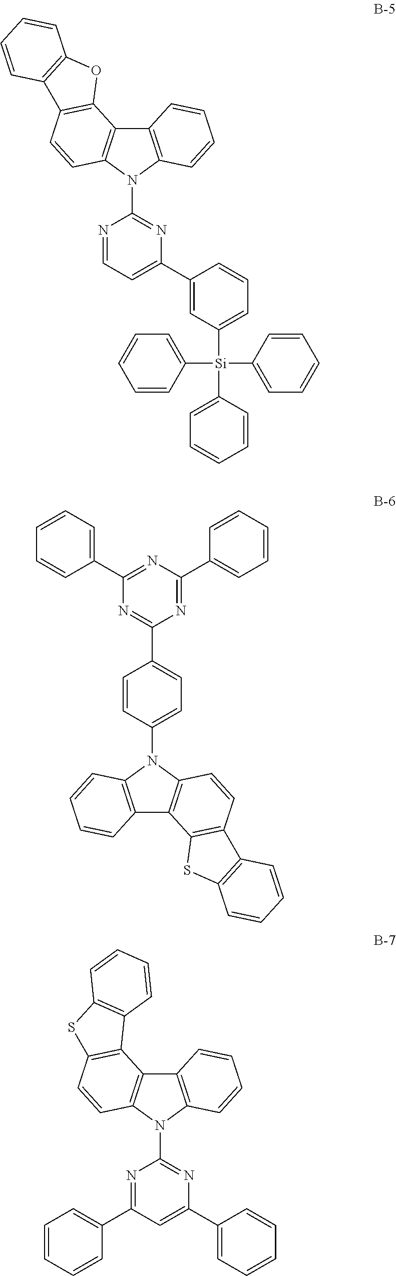 [SMILES: c1ccc([Si](c2ccccc2)(c2ccccc2)c2cccc(-c3ccnc(-n4c5ccccc5c5c6oc7ccccc7c6ccc54)n3)c2)cc1 ; c1ccc(-c2nc(-c3ccccc3)nc(-c3ccc(-n4c5ccccc5c5c6sc7ccccc7c6ccc54)cc3)n2)cc1 ; c1ccc(-c2cc(-c3ccccc3)nc(-n3c4ccccc4c4c5c(ccc43)sc3ccccc35)n2)cc1]

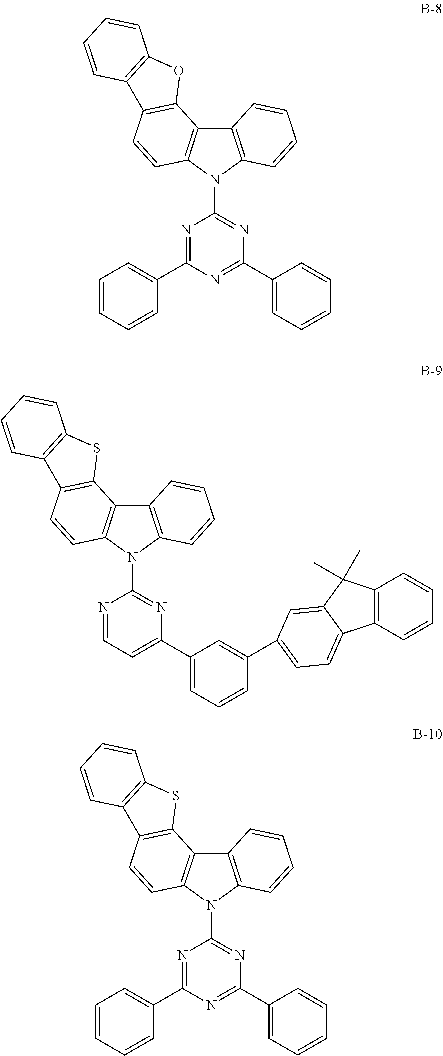 [SMILES: c1ccc(-c2nc(-c3ccccc3)nc(-n3c4ccccc4c4c5oc6ccccc6c5ccc43)n2)cc1 ; CC1(C)c2ccccc2-c2ccc(-c3cccc(-c4ccnc(-n5c6ccccc6c6c7sc8ccccc8c7ccc65)n4)c3)cc21 ; c1ccc(-c2nc(-c3ccccc3)nc(-n3c4ccccc4c4c5sc6ccccc6c5ccc43)n2)cc1]

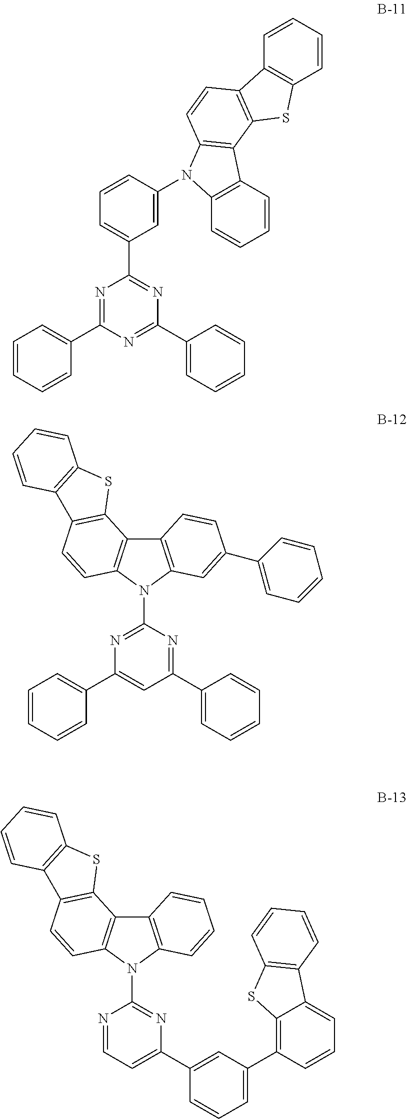

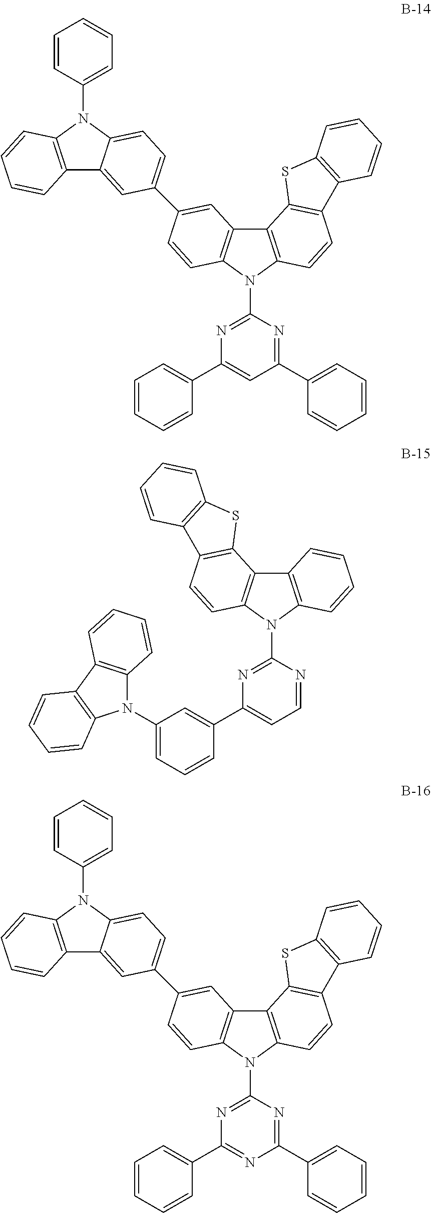

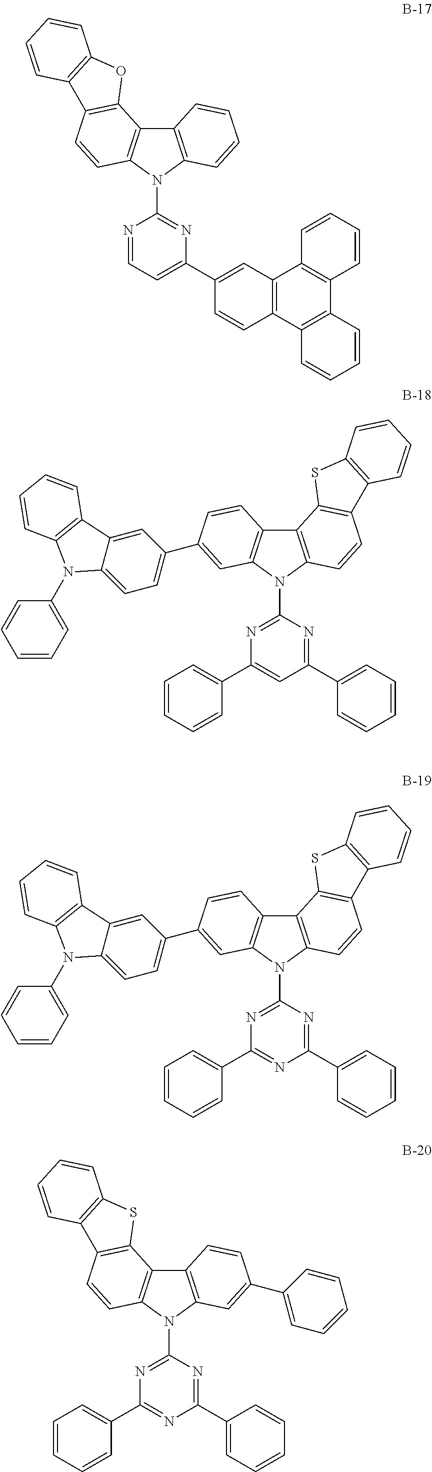 [SMILES: c1ccc2c(c1)oc1c2ccc2c1c1ccccc1n2-c1nccc(-c2ccc3c4ccccc4c4ccccc4c3c2)n1 ; c1ccc(-c2cc(-c3ccccc3)nc(-n3c4cc(-c5ccc6c(c5)c5ccccc5n6-c5ccccc5)ccc4c4c5sc6ccccc6c5ccc43)n2)cc1 ; c1ccc(-c2nc(-c3ccccc3)nc(-n3c4cc(-c5ccc6c(c5)c5ccccc5n6-c5ccccc5)ccc4c4c5sc6ccccc6c5ccc43)n2)cc1 ; c1ccc(-c2ccc3c4c5sc6ccccc6c5ccc4n(-c4nc(-c5ccccc5)nc(-c5ccccc5)n4)c3c2)cc1]

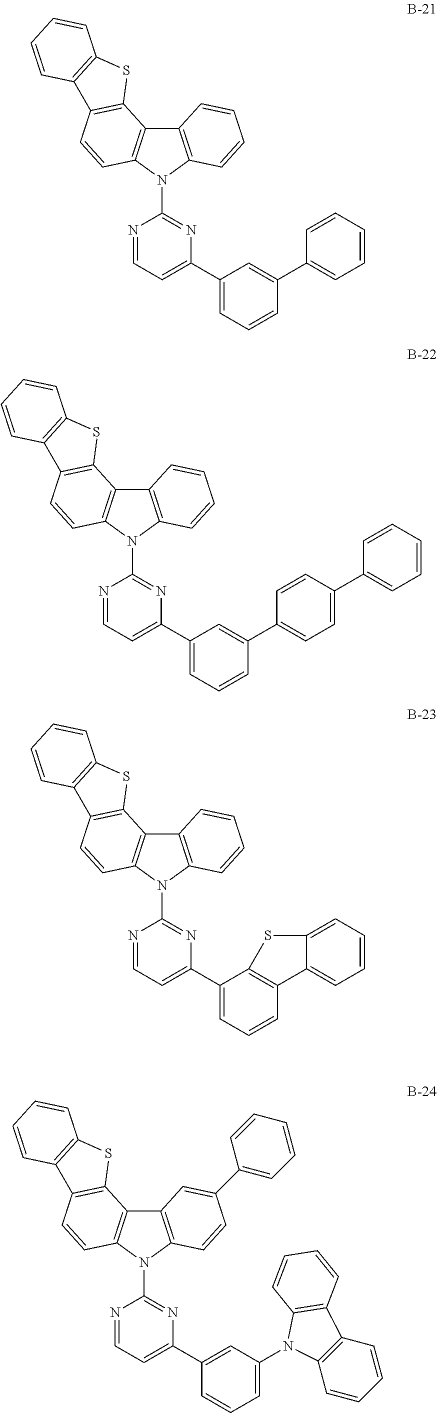 [SMILES: c1ccc(-c2cccc(-c3ccnc(-n4c5ccccc5c5c6sc7ccccc7c6ccc54)n3)c2)cc1 ; c1ccc(-c2ccc(-c3cccc(-c4ccnc(-n5c6ccccc6c6c7sc8ccccc8c7ccc65)n4)c3)cc2)cc1 ; c1ccc2c(c1)sc1c(-c3ccnc(-n4c5ccccc5c5c6sc7ccccc7c6ccc54)n3)cccc12 ; c1ccc(-c2ccc3c(c2)c2c4sc5ccccc5c4ccc2n3-c2nccc(-c3cccc(-n4c5ccccc5c5ccccc54)c3)n2)cc1]

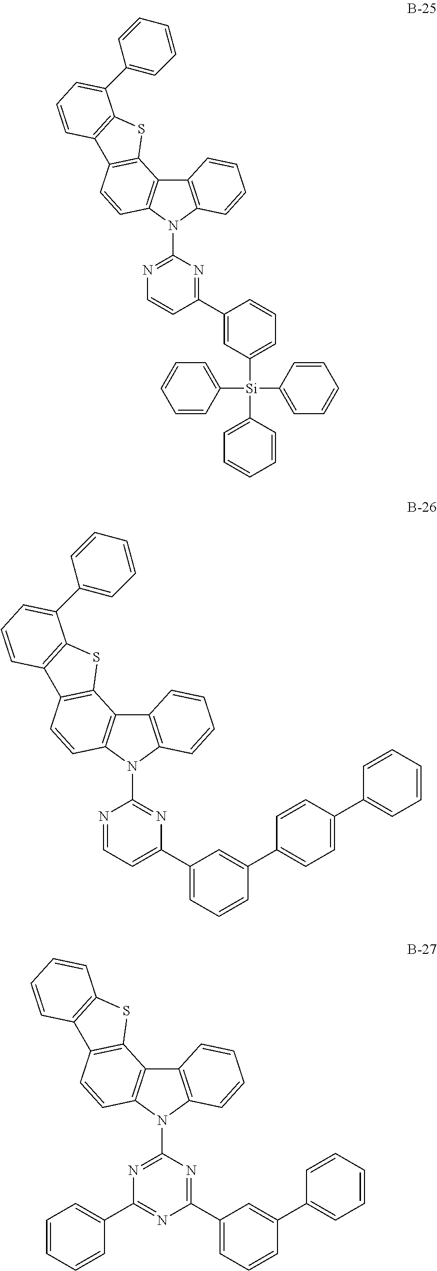 [SMILES: c1ccc(-c2cccc3c2sc2c3ccc3c2c2ccccc2n3-c2nccc(-c3cccc([Si](c4ccccc4)(c4ccccc4)c4ccccc4)c3)n2)cc1 ; c1ccc(-c2ccc(-c3cccc(-c4ccnc(-n5c6ccccc6c6c7sc8c(-c9ccccc9)cccc8c7ccc65)n4)c3)cc2)cc1 ; c1ccc(-c2cccc(-c3nc(-c4ccccc4)nc(-n4c5ccccc5c5c6sc7ccccc7c6ccc54)n3)c2)cc1]

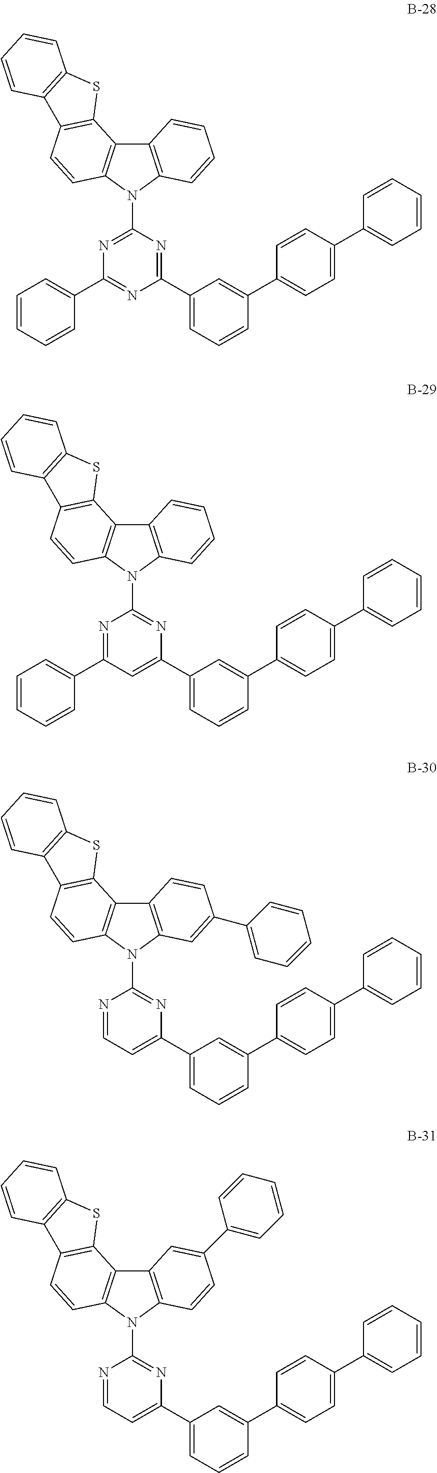 [SMILES: c1ccc(-c2ccc(-c3cccc(-c4nc(-c5ccccc5)nc(-n5c6ccccc6c6c7sc8ccccc8c7ccc65)n4)c3)cc2)cc1 ; c1ccc(-c2ccc(-c3cccc(-c4cc(-c5ccccc5)nc(-n5c6ccccc6c6c7sc8ccccc8c7ccc65)n4)c3)cc2)cc1 ; c1ccc(-c2ccc(-c3cccc(-c4ccnc(-n5c6cc(-c7ccccc7)ccc6c6c7sc8ccccc8c7ccc65)n4)c3)cc2)cc1 ; c1ccc(-c2ccc(-c3cccc(-c4ccnc(-n5c6ccc(-c7ccccc7)cc6c6c7sc8ccccc8c7ccc65)n4)c3)cc2)cc1]

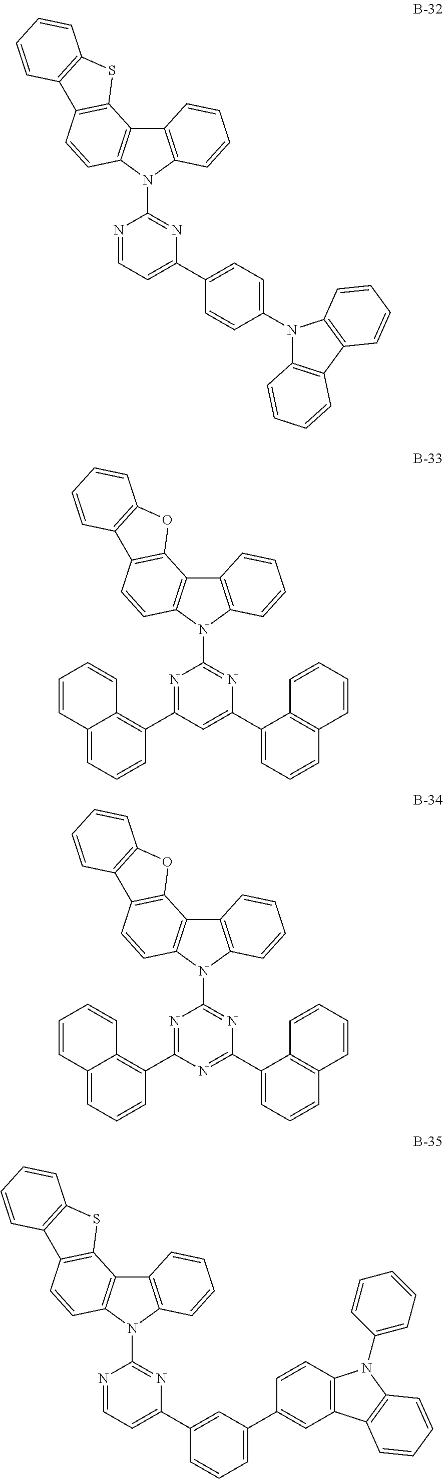

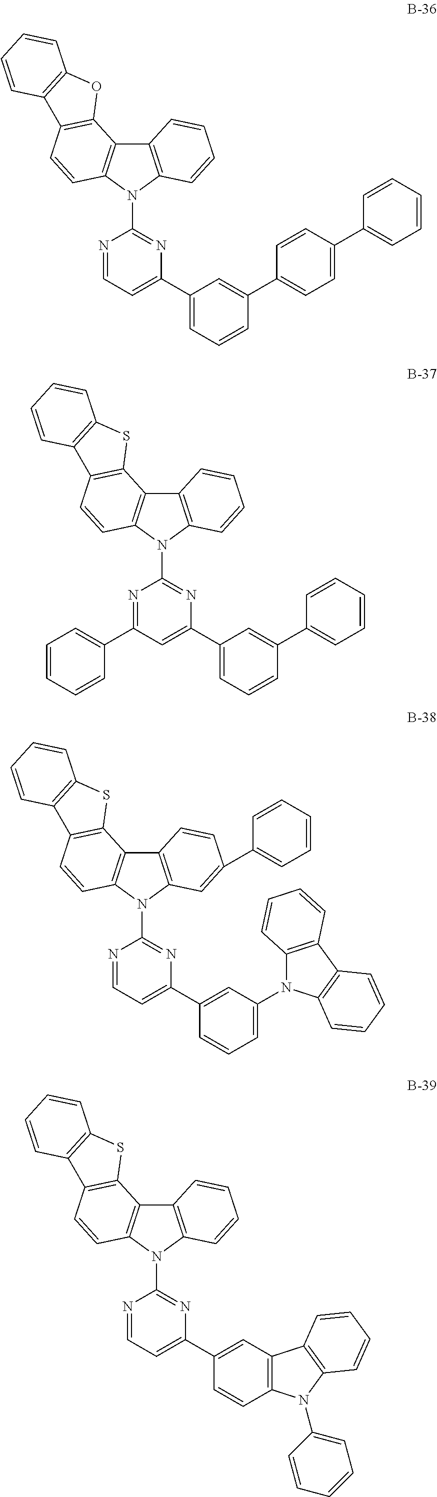 [SMILES: c1ccc(-c2ccc(-c3cccc(-c4ccnc(-n5c6ccccc6c6c7oc8ccccc8c7ccc65)n4)c3)cc2)cc1 ; c1ccc(-c2cccc(-c3cc(-c4ccccc4)nc(-n4c5ccccc5c5c6sc7ccccc7c6ccc54)n3)c2)cc1 ; c1ccc(-c2ccc3c4c5sc6ccccc6c5ccc4n(-c4nccc(-c5cccc(-n6c7ccccc7c7ccccc76)c5)n4)c3c2)cc1 ; c1ccc(-n2c3ccccc3c3cc(-c4ccnc(-n5c6ccccc6c6c7sc8ccccc8c7ccc65)n4)ccc32)cc1]

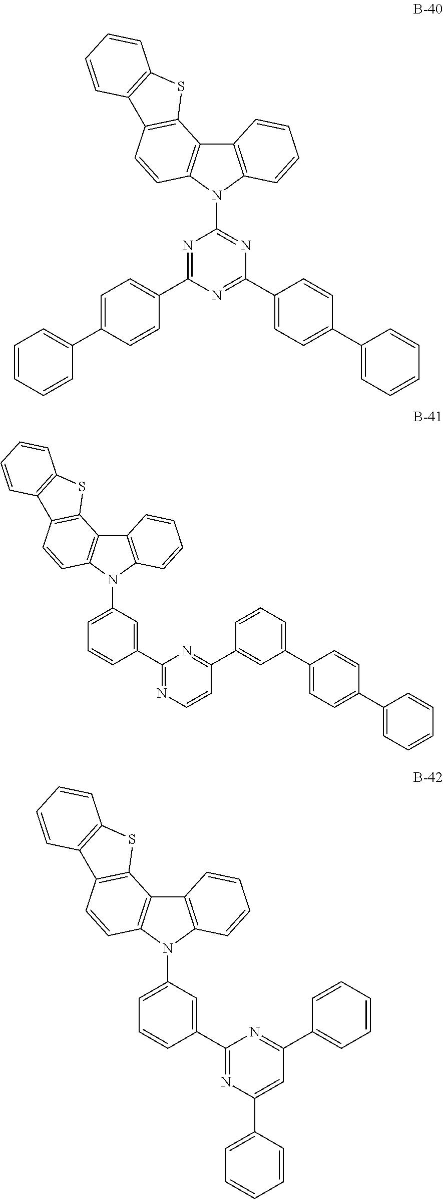 [SMILES: c1ccc(-c2ccc(-c3nc(-c4ccc(-c5ccccc5)cc4)nc(-n4c5ccccc5c5c6sc7ccccc7c6ccc54)n3)cc2)cc1 ; c1ccc(-c2ccc(-c3cccc(-c4ccnc(-c5cccc(-n6c7ccccc7c7c8sc9ccccc9c8ccc76)c5)n4)c3)cc2)cc1 ; c1ccc(-c2cc(-c3ccccc3)nc(-c3cccc(-n4c5ccccc5c5c6sc7ccccc7c6ccc54)c3)n2)cc1]

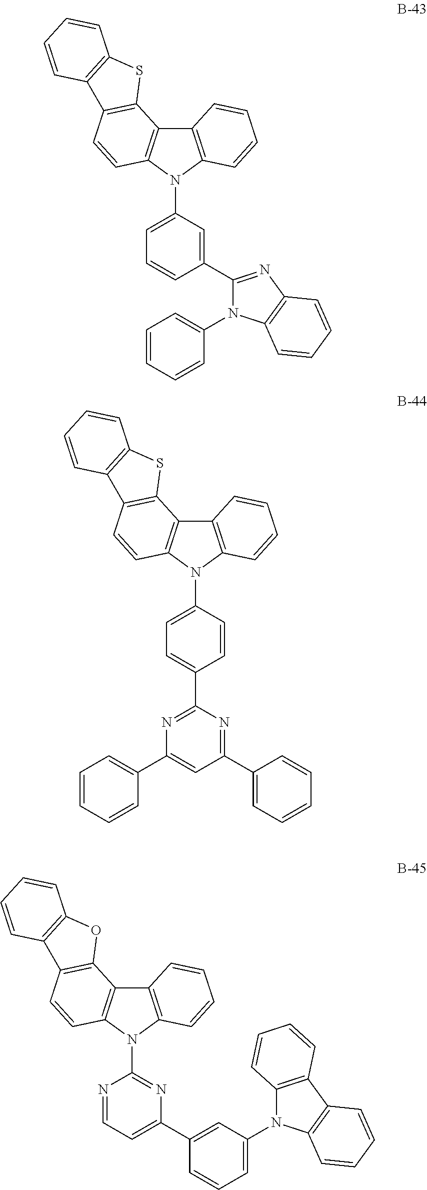 [SMILES: c1ccc(-n2c(-c3cccc(-n4c5ccccc5c5c6sc7ccccc7c6ccc54)c3)nc3ccccc32)cc1 ; c1ccc(-c2cc(-c3ccccc3)nc(-c3ccc(-n4c5ccccc5c5c6sc7ccccc7c6ccc54)cc3)n2)cc1 ; c1cc(-c2ccnc(-n3c4ccccc4c4c5oc6ccccc6c5ccc43)n2)cc(-n2c3ccccc3c3ccccc32)c1]

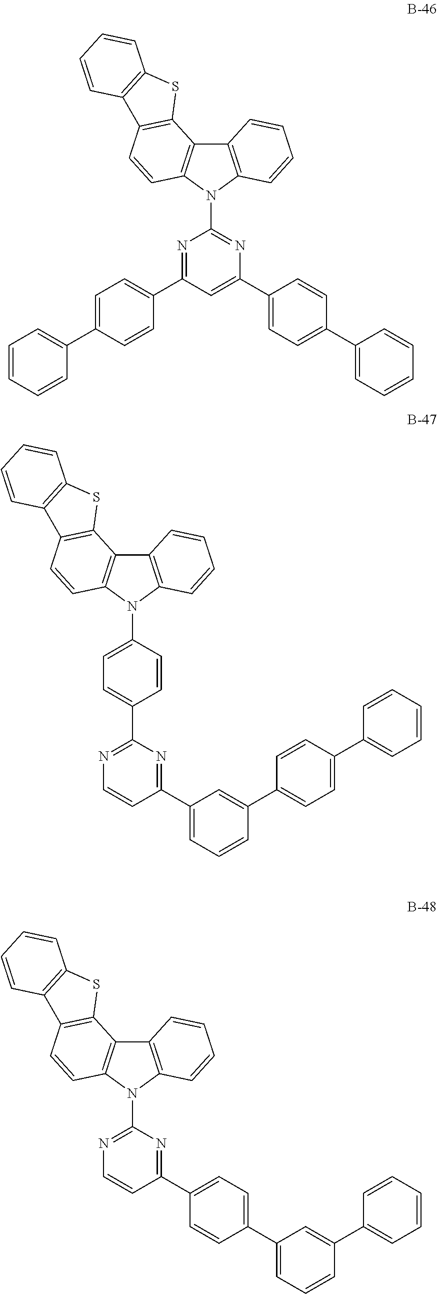 [SMILES: c1ccc(-c2ccc(-c3cc(-c4ccc(-c5ccccc5)cc4)nc(-n4c5ccccc5c5c6sc7ccccc7c6ccc54)n3)cc2)cc1 ; c1ccc(-c2ccc(-c3cccc(-c4ccnc(-c5ccc(-n6c7ccccc7c7c8sc9ccccc9c8ccc76)cc5)n4)c3)cc2)cc1 ; c1ccc(-c2cccc(-c3ccc(-c4ccnc(-n5c6ccccc6c6c7sc8ccccc8c7ccc65)n4)cc3)c2)cc1]

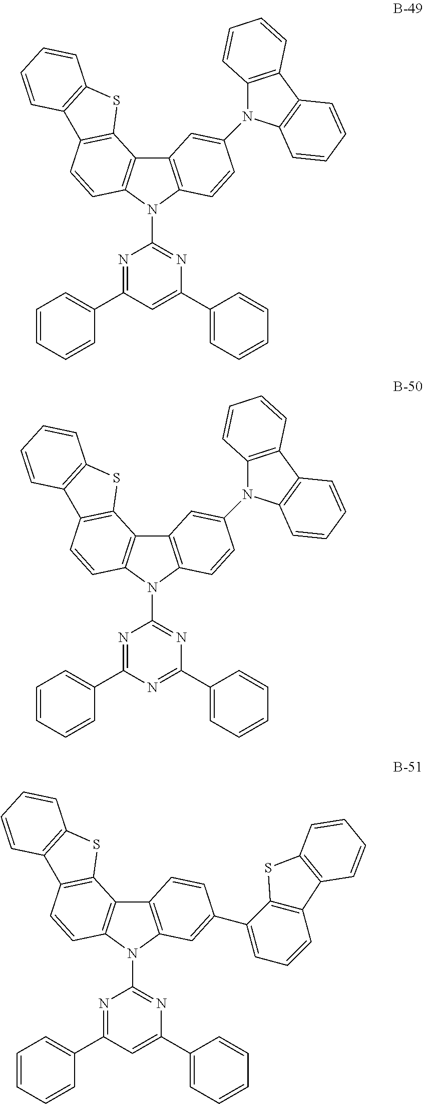

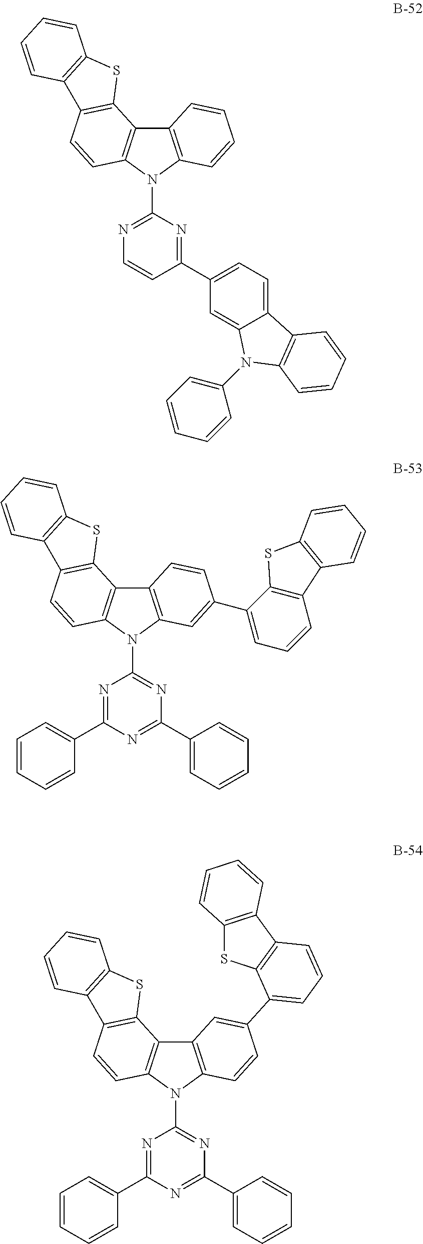 [SMILES: c1ccc(-n2c3ccccc3c3ccc(-c4ccnc(-n5c6ccccc6c6c7sc8ccccc8c7ccc65)n4)cc32)cc1 ; c1ccc(-c2nc(-c3ccccc3)nc(-n3c4cc(-c5cccc6c5sc5ccccc56)ccc4c4c5sc6ccccc6c5ccc43)n2)cc1 ; c1ccc(-c2nc(-c3ccccc3)nc(-n3c4ccc(-c5cccc6c5sc5ccccc56)cc4c4c5sc6ccccc6c5ccc43)n2)cc1]

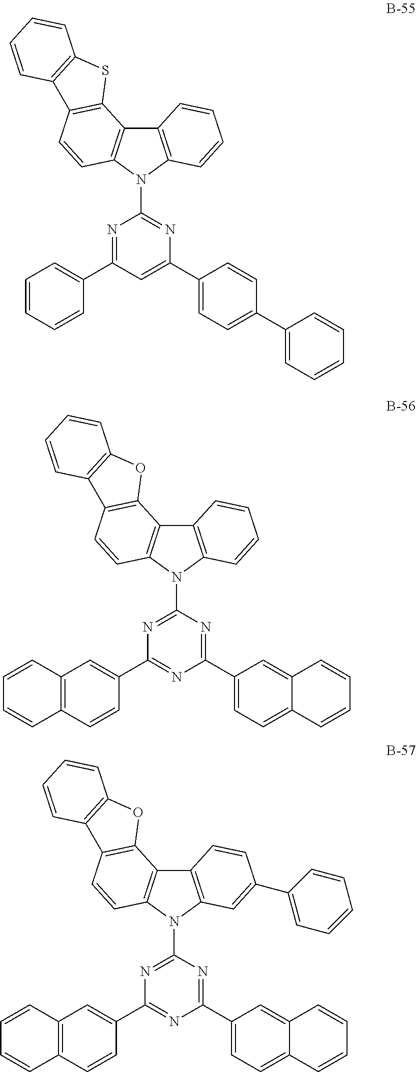

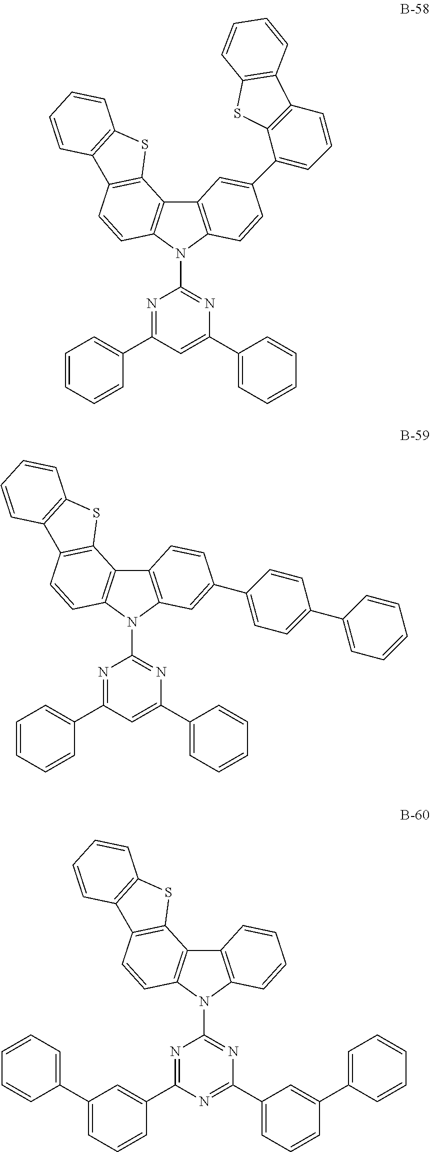 [SMILES: c1ccc(-c2cc(-c3ccccc3)nc(-n3c4ccc(-c5cccc6c5sc5ccccc56)cc4c4c5sc6ccccc6c5ccc43)n2)cc1 ; c1ccc(-c2ccc(-c3ccc4c5c6sc7ccccc7c6ccc5n(-c5nc(-c6ccccc6)cc(-c6ccccc6)n5)c4c3)cc2)cc1 ; c1ccc(-c2cccc(-c3nc(-c4cccc(-c5ccccc5)c4)nc(-n4c5ccccc5c5c6sc7ccccc7c6ccc54)n3)c2)cc1]

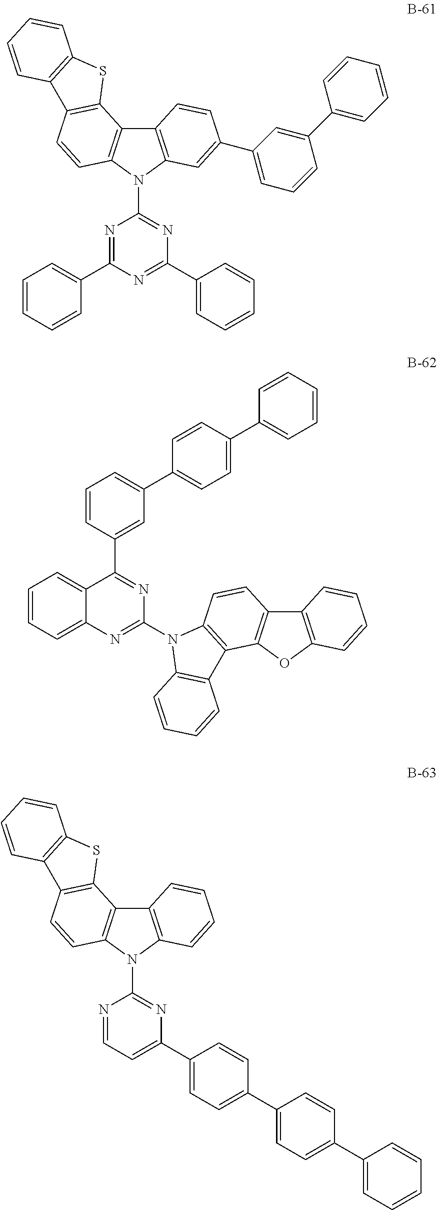 [SMILES: c1ccc(-c2cccc(-c3ccc4c5c6sc7ccccc7c6ccc5n(-c5nc(-c6ccccc6)nc(-c6ccccc6)n5)c4c3)c2)cc1 ; c1ccc(-c2ccc(-c3cccc(-c4nc(-n5c6ccccc6c6c7oc8ccccc8c7ccc65)nc5ccccc45)c3)cc2)cc1 ; c1ccc(-c2ccc(-c3ccc(-c4ccnc(-n5c6ccccc6c6c7sc8ccccc8c7ccc65)n4)cc3)cc2)cc1]

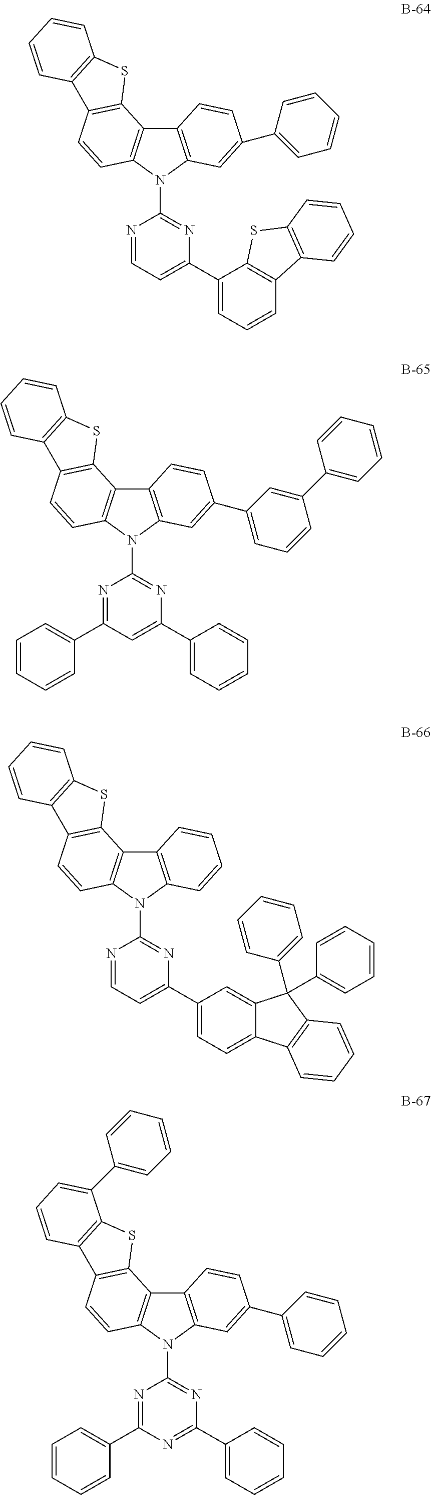 [SMILES: c1ccc(-c2ccc3c4c5sc6ccccc6c5ccc4n(-c4nccc(-c5cccc6c5sc5ccccc56)n4)c3c2)cc1 ; c1ccc(-c2cccc(-c3ccc4c5c6sc7ccccc7c6ccc5n(-c5nc(-c6ccccc6)cc(-c6ccccc6)n5)c4c3)c2)cc1 ; c1ccc(C2(c3ccccc3)c3ccccc3-c3ccc(-c4ccnc(-n5c6ccccc6c6c7sc8ccccc8c7ccc65)n4)cc32)cc1 ; c1ccc(-c2ccc3c4c5sc6c(-c7ccccc7)cccc6c5ccc4n(-c4nc(-c5ccccc5)nc(-c5ccccc5)n4)c3c2)cc1]

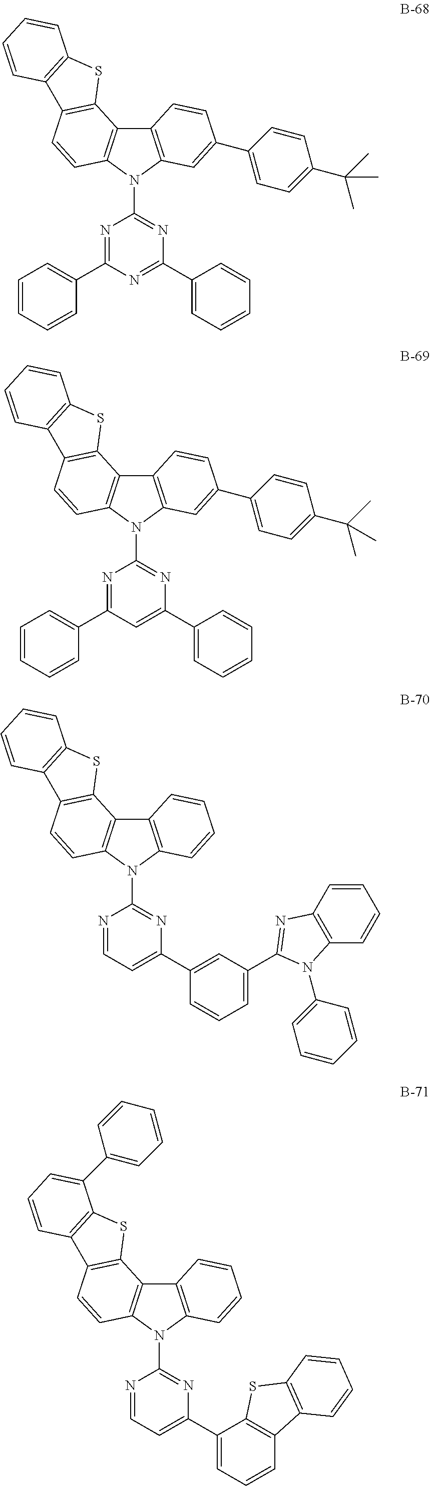

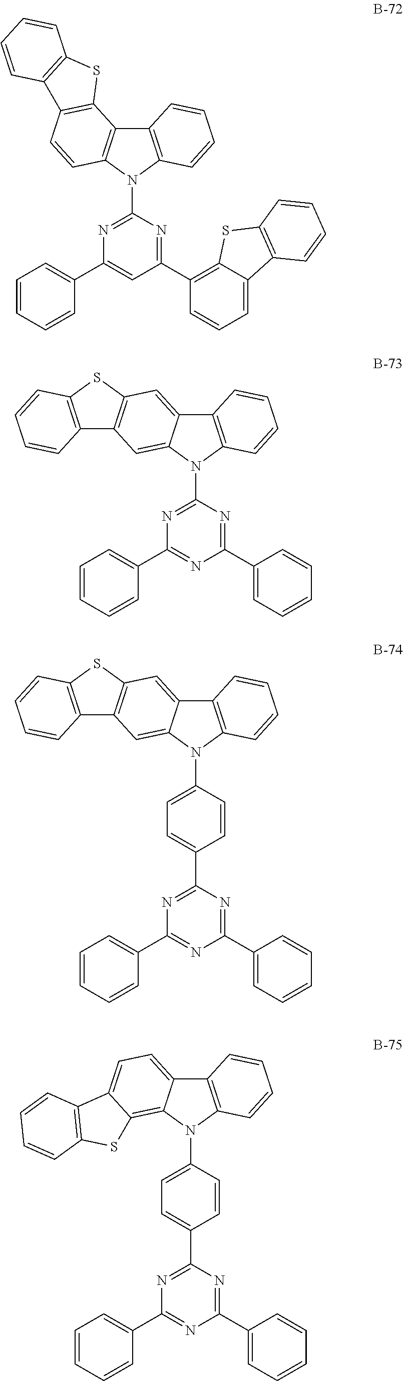 [SMILES: c1ccc(-c2cc(-c3cccc4c3sc3ccccc34)nc(-n3c4ccccc4c4c5sc6ccccc6c5ccc43)n2)cc1 ; c1ccc(-c2nc(-c3ccccc3)nc(-n3c4ccccc4c4cc5sc6ccccc6c5cc43)n2)cc1 ; c1ccc(-c2nc(-c3ccccc3)nc(-c3ccc(-n4c5ccccc5c5cc6sc7ccccc7c6cc54)cc3)n2)cc1 ; c1ccc(-c2nc(-c3ccccc3)nc(-c3ccc(-n4c5ccccc5c5ccc6c7ccccc7sc6c54)cc3)n2)cc1]

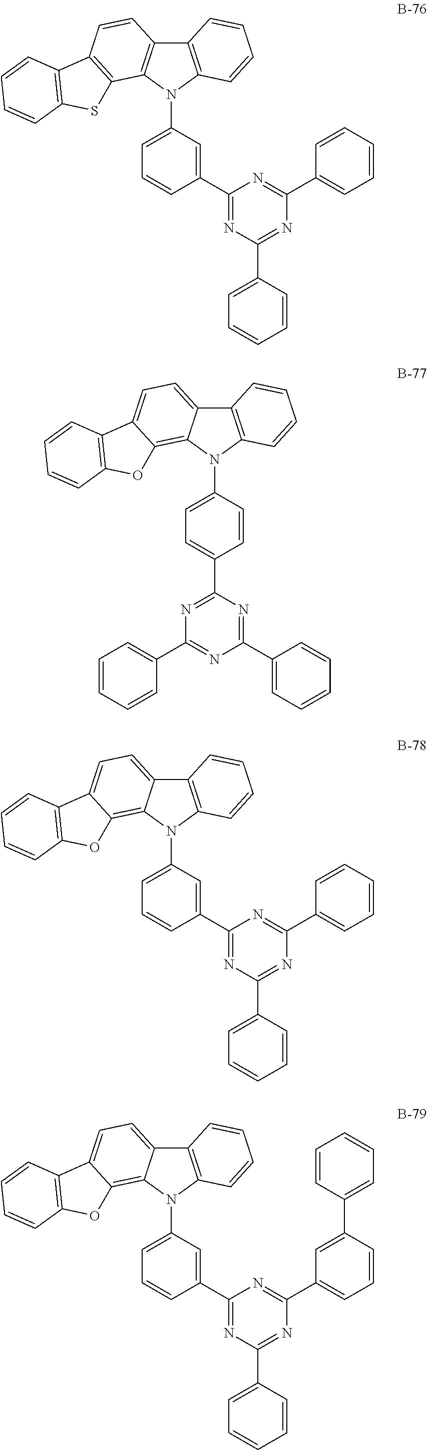

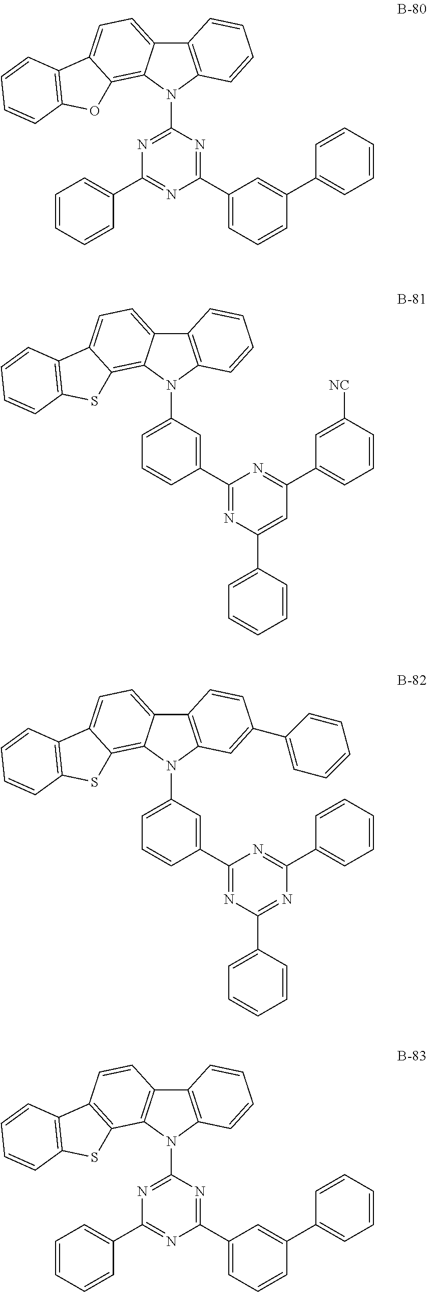 [SMILES: c1ccc(-c2cccc(-c3nc(-c4ccccc4)nc(-n4c5ccccc5c5ccc6c7ccccc7oc6c54)n3)c2)cc1 ; N#Cc1cccc(-c2cc(-c3ccccc3)nc(-c3cccc(-n4c5ccccc5c5ccc6c7ccccc7sc6c54)c3)n2)c1 ; c1ccc(-c2ccc3c4ccc5c6ccccc6sc5c4n(-c4cccc(-c5nc(-c6ccccc6)nc(-c6ccccc6)n5)c4)c3c2)cc1 ; c1ccc(-c2cccc(-c3nc(-c4ccccc4)nc(-n4c5ccccc5c5ccc6c7ccccc7sc6c54)n3)c2)cc1]

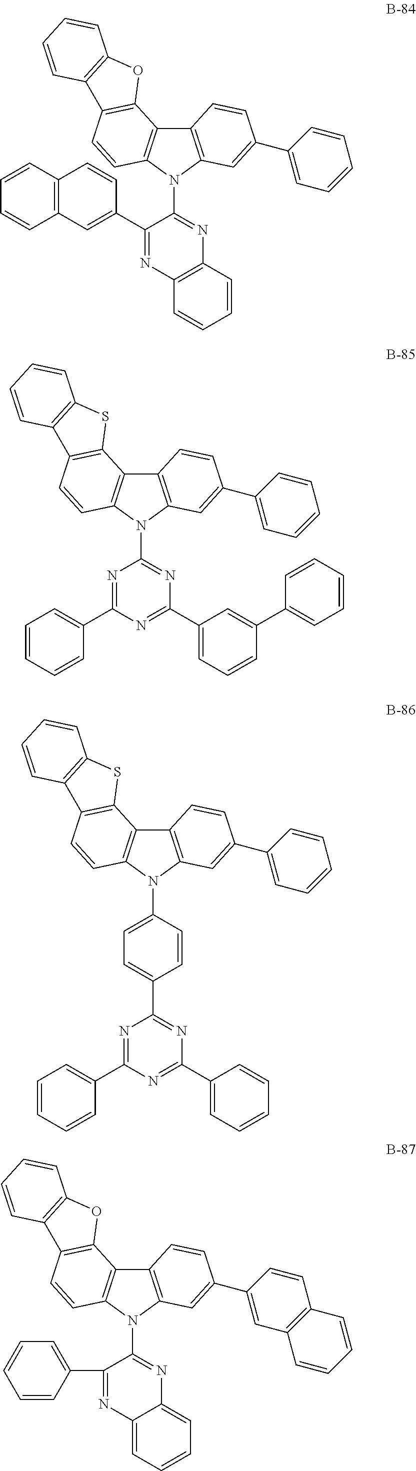

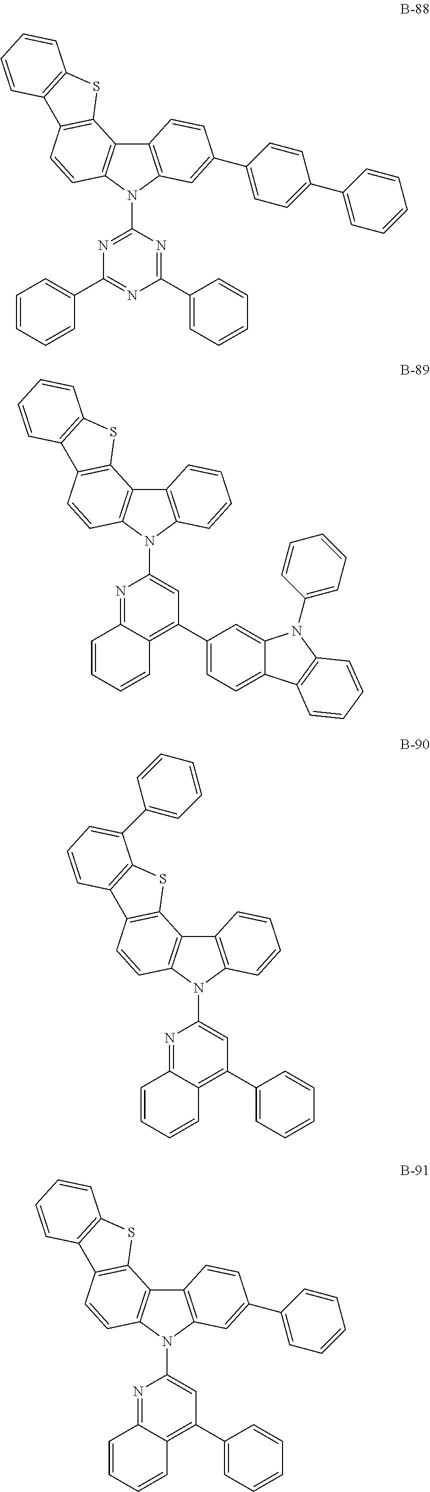

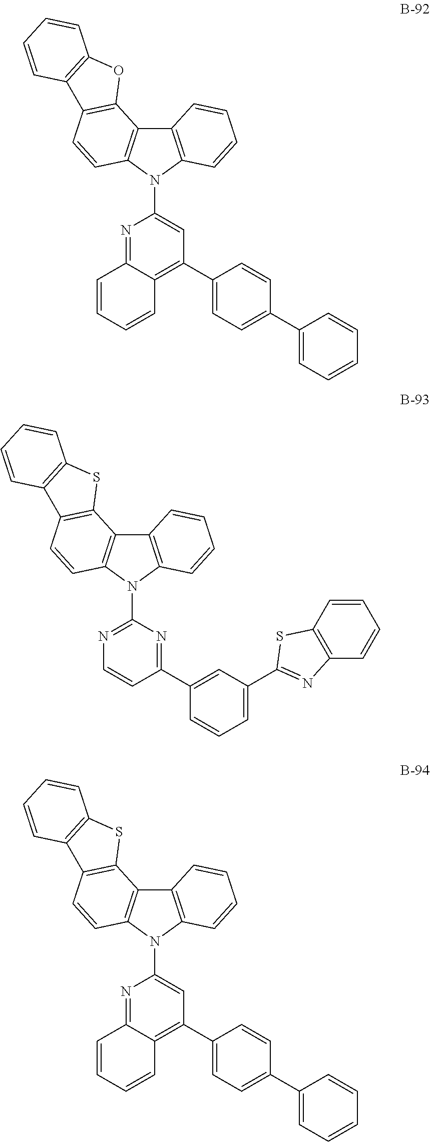 [SMILES: c1ccc(-c2ccc(-c3cc(-n4c5ccccc5c5c6oc7ccccc7c6ccc54)nc4ccccc34)cc2)cc1 ; c1cc(-c2ccnc(-n3c4ccccc4c4c5sc6ccccc6c5ccc43)n2)cc(-c2nc3ccccc3s2)c1 ; c1ccc(-c2ccc(-c3cc(-n4c5ccccc5c5c6sc7ccccc7c6ccc54)nc4ccccc34)cc2)cc1]

The compounds of the present invention represented by formula 1 can be prepared by a synthetic method known to a person skilled in the art. For example, they can be prepared according to the following reaction scheme.

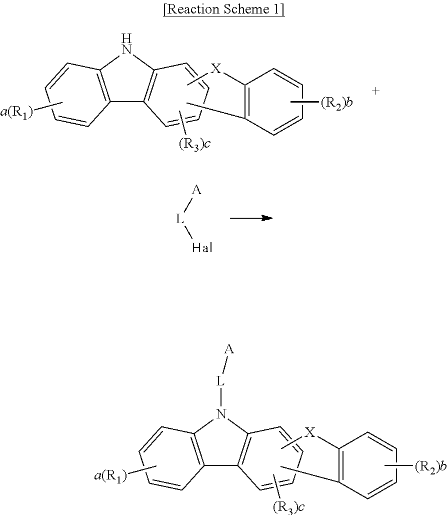

wherein X, L, A, R₁ to R₃, a, b, and c are as defined in formula 1, and Hal represents a halogen.

Another embodiment of the present invention provides the use of the compound represented by formula 1 as an electron buffering material. Preferably, the use may be a use as an electron buffering material for an organic electroluminescent device.

The organic electroluminescent device of the present invention comprises a first electrode; a second electrode facing the first electrode; a light-emitting layer between the first electrode and the second electrode; and an electron transport zone and an electron buffering layer between the light-emitting layer and the second electrode; wherein the electron buffering layer comprises a compound represented by formula 1. When using the compound, the driving voltage, efficiency, and lifespan of the device can be improved.

The electron buffering layer is a layer for solving the problem of a change in luminance caused by the change of a current characteristic of the device when exposed to a high temperature during a process of producing a panel. In order to obtain a similar current characteristic compared to a device without an electron buffering layer, the characteristic of the compound comprised in the electron buffering layer is important. The compound represented by formula 1 forms benzofuro[2,3-a]carbazole or benzothieno[2,3-a]carbazole by a benzofuran or benzothiophene ring being fused to a carbazole derivative. The above structure is rigid by fusing a carbazole to a benzothiophene or benzofuran ring, and thus has almost 0° of dihedral angle. Accordingly, relevant bulky groups have great intermolecular π-orbital overlap, and thus intermolecular charge transition becomes easier. It is considered that if the intermolecular π-π stacking is reinforced, fast electron current characteristic can be achieved through a coplanar structure. In contrast, when carbazole and a dibenzothiophene or dibenzofuran ring are linked via a methyl, its dihedral angle has a deviation of about 36° which provides relatively random molecular orientation, and thereby resulting in decrease of electron current characteristic and efficiency. Therefore, the compound according to the present invention can highly contribute to a low driving voltage and an improvement in the efficiency and lifespan of an organic electroluminescent device. This improvement of the device characteristics has a great effect on the improvement of the performance in the process of producing panels.

By interposing the electron buffering layer between the light-emitting layer and the second electrode in the organic electroluminescent device comprising the first and second electrodes and the light-emitting layer, an electron injection can be controlled by electron affinity LUMO energy level of the electron buffering layer.

In the organic electroluminescent device of the present invention, the LUMO energy level of the electron buffering layer may be higher than the LUMO energy level of the host compound. Specifically, the difference in the LUMO energy levels between the electron buffering layer and the host compound may be 0.3 eV or less. For example, the LUMO energy levels of the electron buffering layer and the host compound may be 1.9 eV and 1.6 eV, respectively, and thus the difference in the LUMO energy levels may be 0.3 eV. Although the LUMO barrier between the host compound and the electron buffering layer can cause an increase in the driving voltage, electrons can be more easily transferred to the host compound due to the existence of the compound of formula 1 comprised in the electron buffering layer, compared to other compounds. Therefore, the organic electroluminescent device of the present invention can have low driving voltage, high luminous efficiency, and long lifespan. Herein, specifically, the LUMO energy level of the electron buffering layer may indicate the LUMO energy level of the compound of formula 1 comprised in the electron buffering layer.

In the organic electroluminescent device of the present invention, an electron transport zone means a zone in which electrons are transported from the second electrode to the light-emitting layer. The electron transport zone can comprise an electron transport compound, a reductive dopant, or a combination thereof. The electron transport compound can be at least one selected from the group comprising oxazole-based compounds, isoxazole-based compounds, triazole-based compounds, isothiazole-based compounds, oxadiazole-based compounds, thiadiazole-based compounds, perylene-based compounds, anthracene-based compounds, aluminum complexes, and gallium complexes. The reductive dopant can be at least one selected from the group consisting of alkali metals, alkali metal compounds, alkaline-earth metals, rare earth metals, halides thereof, oxides thereof, and complexes thereof. In addition, the electron transport zone can comprise an electron transport layer, an electron injection layer, or both of them. The electron transport layer and the electron injection layer can each be composed of two or more layers. The LUMO energy level of the electron buffering layer may be higher or lower than the LUMO energy level of the electron transport zone. For example, the electron buffering layer and the electron transport zone may have LUMO energy levels of 1.9 eV and 1.8 eV, respectively, and the difference between them in LUMO energy levels may be 0.1 eV. When the electron buffering layer has the LUMO energy level as in said numerical range, electrons can be easily injected to the light-emitting layer through the electron buffering layer. The LUMO energy level of the electron transport zone may be 1.7 eV or more, or 1.9 eV or more.

Specifically, the LUMO energy level of the electron buffering layer may be higher than those of the host compound and the electron transport zone. For example, the LUMO energy levels may have the following relationship: the electron buffering layer>the electron transport zone>the host compound. According to the aforementioned LUMO relationship, electrons are trapped between the light-emitting layer and the electron buffering layer, which inhibits an injection of electrons, and thus can cause an increase in driving voltage. However, the electron buffering layer comprising the compound of formula 1 can easily transport electrons to the light-emitting layer, and thus the organic electroluminescent device of the present invention can have low driving voltage, high luminous efficiency, and long lifespan.

The LUMO energy level can be easily measured by known various methods. Generally, cyclic voltametry or ultraviolet photoelectron spectroscopy (UPS) is used. Therefore, one skilled in the art can easily understand and determine the electron buffering layer, host material, and electron transport zone which satisfy the aforementioned relationship for the LUMO energy levels, so that he/she can easily practice the invention. The HOMO energy level can be easily measured in the same manner as the LUMO energy level.

The layers of the organic electroluminescent device of the present invention can be formed in the order of light-emitting layer, electron buffering layer, electron transport zone, and second electrode, or in the order of light-emitting layer, electron transport zone, electron buffering layer, and second electrode.

In addition, the organic electroluminescent device of the present invention may further comprise a hole injection layer, a hole transport layer, or both between the first electrode and the light-emitting layer.

Hereinafter, referring to FIG. 1, the structure of an organic electroluminescent device, and a method for preparing it will be described in detail.

The organic electroluminescent device of FIG. 1 is only an embodiment to be explained clearly, and the present invention should not be limited to the embodiment but can be varied to another mode. For example, an optional component of the organic electroluminescent device of FIG. 1 besides a light-emitting layer and an electron buffering layer can be omitted such as the hole injection layer. In addition, an optional component can be further added. Examples of the further added optional component are impurity layers such as n-doping layer and p-doping layer. Moreover, the organic electroluminescent device can emit light from both sides by placing a light-emitting layer each in both sides in between the impurity layers. The light-emitting layers of both sides can emit different colors. In addition, the first electrode can be a transparent electrode and the second electrode can be a reflective electrode so that the organic electroluminescent device can be a bottom emission type, and the first electrode can be a reflective electrode and the second electrode can be a transparent electrode so that the organic electroluminescent device can be a top emission type. Also, a cathode, an electron transport layer, a light-emitting layer, a hole transport layer, a hole injection layer, and an anode can be sequentially stacked on a substrate to be an inverted organic electroluminescent device.

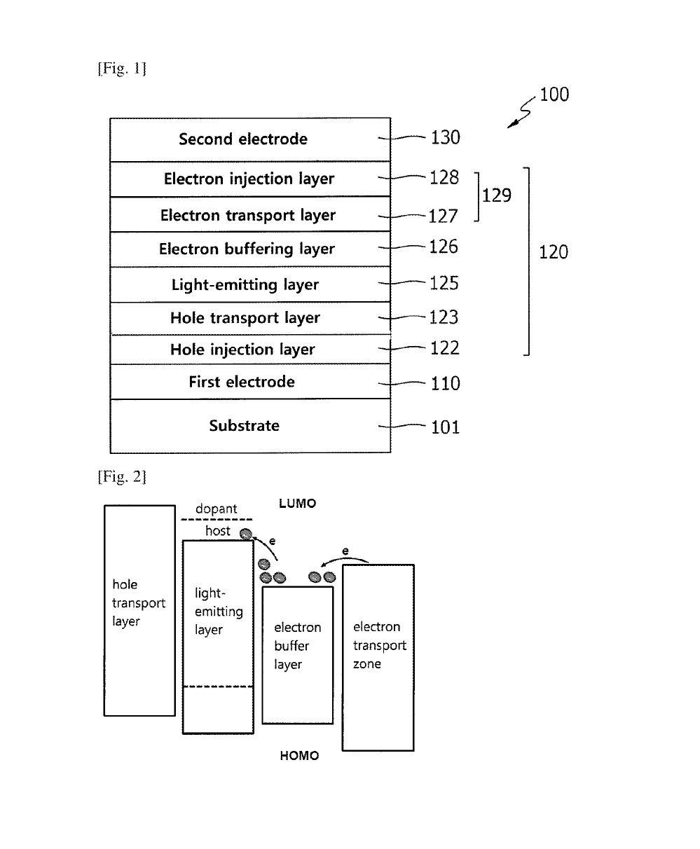

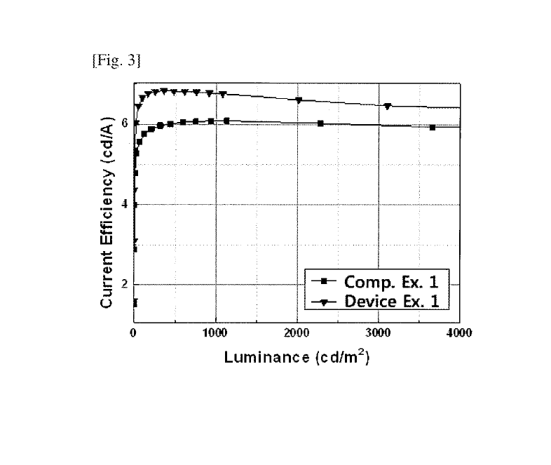

FIG. 2 is an energy band diagram of a hole transport layer, a light-emitting layer, an electron buffering layer, and an electron transport zone of an organic electroluminescent device according to one embodiment of the present invention.

In FIG. 2, a hole transport layer (123), a light-emitting layer (125), an electron buffering layer (126), and an electron transport zone (129) are sequentially stacked, and electrons are injected from the cathode to the light-emitting layer (125) through the electron transport zone (129) and the electron buffering layer (126).

Hereinafter, the organic electroluminescent compound, the preparation method of the compound, and the luminescent properties of the device comprising an electron buffering material comprising the compound will be explained in detail with reference to the following examples.

### Example 1: Preparation of Compound B-3

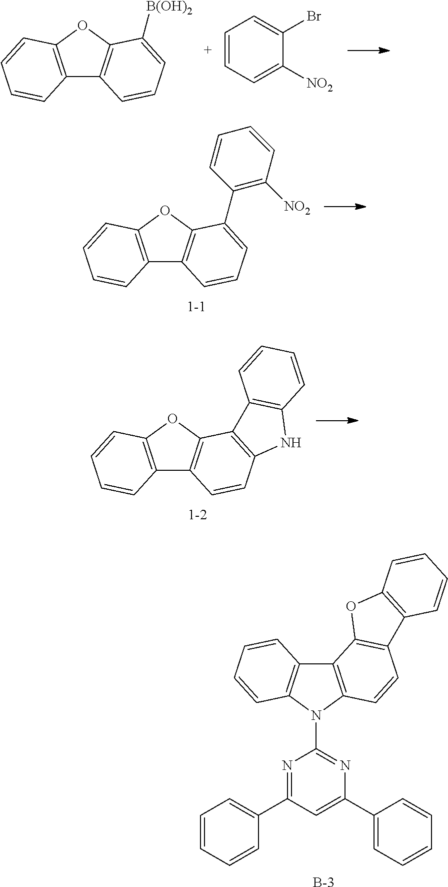

Preparation of Compound 1-1

After mixing 1-bromo-2-nitrobenzene (39 g, 0.19 mol), dibenzo[b,d]furan-4-yl boronic acid (45 g, 0.21 mol), Pd(PPh₃)₄ (11.1 g, 0.0096 mol), 2 M K₂CO₃ aqueous solution 290 mL, EtOH 290 mL, and toluene 580 mL, the reactant mixture was stirred for 4 hours while heated to 120° C. After the reaction is completed, the mixture was washed with distilled water, and extracted with EA. The extracted organic layer was dried with anhydrous MgSO₄, and the solvent was removed with a rotary evaporator. The residue was purified by column chromatography to obtain compound 1-1 (47 g, 85%).

Preparation of Compound 1-2

After mixing compound 1-1 (47 g, 0.16 mol), triethylphosphite 600 mL, and 1,2-dichlorobenzene 300 mL, the reactant mixture was heated to 150° C. and stirred for 12 hours. After the reaction is completed, unreacted triethylphosphite and 1,2-dichlorobenzene were removed using a distillation apparatus. The remaining mixture was washed with distilled water, and extracted with EA. The extracted organic layer was dried with anhydrous MgSO₄, and the solvent was removed with a rotary evaporator. The residue was purified by column chromatography to obtain compound 1-2 (39 g, 81%).

Preparation of Compound B-3

NaH (1.9 mg, 42.1 mmol) was dissolved in dimethylformamide (DMF) and stirred. After dissolving compound 1-2 (7 g, 27.2 mmol) in DMF, the mixture was added to the NaH solution above, and the mixture was stirred for 1 hour. 2-chloro-4,6-dimethylpyrimidine (8.7 g, 32.6 mmol) was dissolved in DMF, and the reactant above which was stirred for 1 hour was added thereto, and the mixture was stirred at room temperature for 24 hours. After the reaction is completed, the obtained solid was filtered. The filtrate was washed with ethyl acetate, and purified by column chromatography to obtain the objective compound B-3 (3.5 g, 25%).

### Example 2: Preparation of Compound B-10

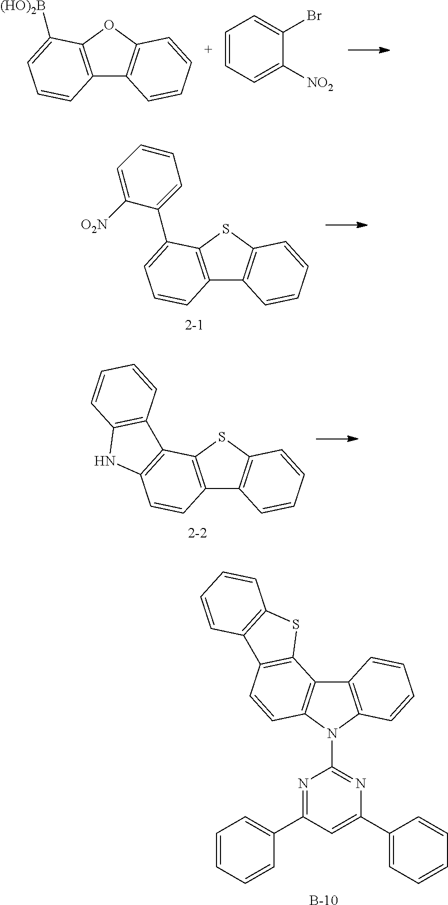

Preparation of Compound 2-1

Compound 2-1 (10 g, 32.74 mmol, 74.68%) was obtained by the synthetic method of compound 1-1 using dibenzo[b,d]thiophen-4-yl boronic acid (10 g, 43.84 mmol).

Preparation of Compound 2-2

Compound 2-2 (7 g, 25.60 mmol, 78.19%) was obtained by the synthetic method of compound 1-2 using compound 2-1 (10 g, 32.74 mmol).

Preparation of Compound B-10

The objective compound B-10 (5.6 g, 40%) was obtained by the synthetic method of compound B-3 using compound 2-2 (7 g, 25.6 mmol) and 2-chloro-4,6-diphenyl-1,3,5-triazine (8.7 g, 32.6 mmol).

### Example 3: Preparation of Compound B-22

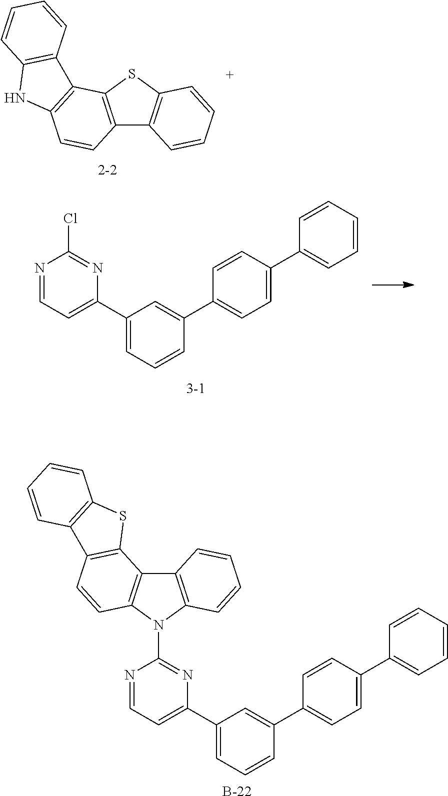

The objective compound B-22 (5.3 g, 49%) was obtained by the synthetic method of compound B-3 using compound 2-2 (7 g, 25.6 mmol) and compound 3-1 (8.2 g, 32.6 mmol).

Compounds B-1 to B-72 were synthesized by the same method as Examples 1 to 3 above. Specific property data of the representative compounds therefrom are listed in Table 1 as below:

TABLE 1

| | Yield | Melting | UV | PL | MS/EIMS |
|---|---|---|---|---|---|
| Compound | (%) | point (° C.) | (nm) | (nm) | (found) |
| 3 | 25 | 260 | 358 | 471 | 488.5 |
| 4 | 30 | 259 | 336 | 463 | 686.9 |
| 6 | 26 | 350 | 356 | 429 | 581.7 |
| 7 | 46 | 225 | 338 | 482 | 504.3 |
| 8 | 78 | 312 | 344 | 385 | 489.5 |
| 9 | 67 | 249 | 324 | 458 | 610.7 |
| 10 | 40 | 324 | 352 | 482 | 505.7 |
| 11 | 45 | 255 | 334 | 451 | 581.7 |
| 12 | 89 | 275 | 320 | 456 | 580.7 |
| 13 | 72 | 267 | 334 | 459 | 610.7 |
| 15 | 46 | 270 | 344 | 471 | 593.7 |
| 18 | 42 | 288 | 370 | 475 | 745.9 |
| 19 | 28 | 323 | N/A | N/A | 746.8 |
| 20 | 39 | 320 | 325 | 516 | 581.7 |
| 21 | 38 | 198 | 317 | 461 | 504.6 |
| 22 | 49 | 274 | 322 | 491 | 580.7 |
| 24 | 49 | 284 | 368 | 474 | 669.8 |
| 25 | 23 | 270 | 324 | 456 | 763 |
| 26 | 26 | 245 | 300 | 460 | 656.8 |
| 27 | 52 | 241 | 294 | 464 | 581.7 |
| 28 | 42 | 328 | 343 | 481 | 656.8 |
| 29 | 32 | 294 | 296 | 467 | 655.2 |
| 31 | 34 | 294 | N/A | N/A | 656.8 |
| 32 | 60 | 280 | 294 | 468 | 593.7 |
| 34 | 46 | 324 | 324 | 495 | 589.7 |
| 35 | 82 | 250 | 356 | 448 | 669.8 |
| 38 | 30 | 293 | 344 | 469 | 669.8 |
| 39 | 23 | 238 | 362 | 429 | 593.7 |
| 40 | 44 | 357 | 322 | 460 | 655.8 |
| 44 | 48 | 278 | 344 | 395 | 580.7 |
| 47 | 48 | 221 | 334 | 396 | 656.8 |
| 49 | 16 | 347 | 324 | 525 | 669.9 |
| 50 | 34 | 410 | 258 | 324 | 670.8 |
| 51 | 36 | 300 | 258 | 487 | 686.9 |
| 52 | 57 | 261 | 344 | 431 | 593.7 |
| 55 | 23 | 300 | 336 | 458 | 580.7 |
| 64 | 24 | 275 | 344 | 467 | 610.8 |
| 67 | 50 | 305 | 350 | 502 | 656.8 |
| 68 | 66 | 305 | 306 | 407 | 637.8 |
| 69 | 22 | 238 | 304 | 465 | 636.8 |
| 70 | 27 | 274 | 308 | 463 | 620.7 |

### Comparative Example 1: Preparation of a Blue-Emitting OLED in which an Electron Buffering Layer is not Comprised

An OLED was produced as follows. A transparent electrode indium tin oxide (ITO) thin film (15 Q/sq) on a glass substrate for an OLED (Geomatec) was subjected to an ultrasonic washing with trichloroethylene, acetone, and distilled water, sequentially, and then was stored in isopropanol. The ITO substrate was then mounted on a substrate holder of a vacuum vapor depositing apparatus. N⁴,N^{4′}-diphenyl-N⁴,N^{4′}-bis(9-phenyl-9H-carbazol-3-yl)-[1,1′-biphenyl]-4,4′-diamine (compound HI-1) was introduced into a cell of the vacuum vapor depositing apparatus, and then the pressure in the chamber of said apparatus was controlled to 10⁻⁶ torr. Thereafter, an electric current was applied to the cell to evaporate the above introduced material, thereby forming a first hole injection layer having a thickness of 60 nm on the ITO substrate. 1,4,5,8,9,12-hexaazetriphenylene-hexacarbonitrile (HAT-CN) (compound HI-2) was then introduced into another cell of the vacuum vapor depositing apparatus, and was evaporated by applying an electric current to the cell, thereby forming a second hole injection layer having a thickness of 5 nm on the first hole injection layer. N-([1,1′-biphenyl]-4-yl)-9,9-dimethyl-N-(4-(9-phenyl-9H-carbazol-3-yl)phenyl)-9H-fluoren-2-amine (compound HT-1) was then introduced into another cell of the vacuum vapor depositing apparatus, and was evaporated by applying an electric current to the cell, thereby forming a first hole transport layer having a thickness of 20 nm on the second hole injection layer. Thereafter, compound HT-2 was introduced into another cell of the vacuum vapor depositing apparatus, and was evaporated by applying an electric current to the cell, thereby forming a second hole transport layer having a thickness of 5 nm on the first hole transport layer. Thereafter, compound BH-1 was introduced into one cell of the vacuum vapor depositing apparatus, as a host material, and compound BD-1 was introduced into another cell as a dopant. The two materials were evaporated at different rates, so that the dopant was deposited in a doping amount of 2 wt % based on the total amount of the host and dopant to form a light-emitting layer having a thickness of 20 nm on the hole transport layer. 2-(4-(9,10-di(naphthalen-2-yl)anthracen-2-yl)phenyl)-1-phenyl-1H-benzo[d]imidazole (compound ETL-1) was then introduced into one cell, and lithium quinolate was introduced into another cell. The two materials were evaporated at the same rate, so that they were respectively deposited in a doping amount of 50 wt % to form an electron transport layer having a thickness of 35 nm on the light-emitting layer. After depositing lithium quinolate (compound EIL-1) as an electron injection layer having a thickness of 2 nm on the electron transport layer, an Al cathode having a thickness of 80 nm was then deposited by another vacuum vapor deposition apparatus on the electron injection layer. Thus, an OLED was produced. All the materials used for producing the OLED device were those purified by vacuum sublimation at 10⁻⁶ torr.

FIG. 3 shows a graph illustrating current efficiency versus luminance of the prepared organic electroluminescent device. In addition, driving voltage at 1,000 nit of luminance, luminous efficiency, CIE color coordinate, and the time period for the luminance to decrease from 100% to 90% at 2,000 nit and constant current are shown in Table 2 below.

### Device Examples 1 to 6: Preparation of a Blue-Emitting OLED According to the Present Invention

OLEDs were produced and evaluated in the same manner as in Comparative Example 1, except that the thickness of the electron transport layer was 30 nm, and an electron buffering layer having a thickness of 5 nm was interposed between the light-emitting layer and the electron transport layer. FIG. 3 shows a graph illustrating current efficiency versus luminance of the prepared organic electroluminescent device. In addition, evaluation results of the devices prepared in Device Examples 1 to 6 are shown in Table 2 below.

### Comparative Example 2: Preparation of a Blue-Emitting OLED Comprising an Electron Buffering Layer of a Conventional Electron Buffering Material

An OLED was produced and evaluated in the same manner as in Example 1, except that BF-1 was used for the electron buffering material. Evaluation results of the device prepared in Comparative Example 2 is shown in Table 2 below.

TABLE 2

| | | | | Color | Color | |
|---|---|---|---|---|---|---|
| | Electron | | | coor- | coor- | Life- |
| | buffering | Voltage | Efficiency | dinate | dinate | span |
| | layer | (V) | (cd/A) | (x) | (y) | (hr) |
| Comparative | — | 4.4 | 6.1 | 140 | 100 | 27.4 |
| Example 1 | | | | | | |
| Comparative | BF-1 | 4.6 | 5.9 | 139 | 96 | 42.5 |
| Example 2 | | | | | | |
| Device | B-78 | 4.4 | 6.8 | 139 | 95 | 33.1 |
| Example 1 | | | | | | |
| Device | B-80 | 4.5 | 6.6 | 139 | 96 | 30.7 |
| Example 2 | | | | | | |
| Device | B-77 | 4.5 | 6.6 | 139 | 95 | 37.1 |
| Example 3 | | | | | | |
| Device | B-79 | 4.5 | 6.4 | 139 | 94 | 37.4 |
| Example 4 | | | | | | |
| Device | B-76 | 4.5 | 6.3 | 139 | 96 | 37.5 |
| Example 5 | | | | | | |
| Device | B-85 | 4.2 | 7.1 | 139 | 95 | 29.0 |
| Example 6 | | | | | | |

From Table 2 above, it is recognized that due to the fast electron current characteristic of the electron buffering material of the present invention, the devices of Device Examples 1 to 6 show higher efficiency and longer lifespan than those of Comparative Example 1 in which an electron buffering layer is not comprised. In addition, upon comparing Device Example 3 with Comparative Example 2, carbazole and dibenzothiophene is linked via phenylene in compound BF-1 which is used in Comparative Example 2 that the dihedral angle is relatively large, and thus showed a higher voltage and low efficiency due to relatively rough electron injection. Instead, electron current was inhibited in Comparative Example 2 and showed an improvement in lifespan characteristics due to decrease in interfacial stress occurred from relatively low distribution of excitons that used to be mainly formed in the HTL/EML interface. This feature is not preferable in blue fluorescent device requiring high efficiency.

[Analysis of Characteristics]

In order to prove that the efficiency difference of the above devices (using compounds BF-1 and B-77) is based on stacking effects according to molecular arrangements, the difference between the electron buffering materials was confirmed by comparing dipole moment values according to density functional theory (DFT) calculation. As a result, compound B-77 was found to have a lower dipole moment value than compound BF-1. Low dipole moment value means that a compound has planar molecular arrangements, which contributes to an improvement in charge carrier injection characteristics. This is confirmed by the references [Appl. Phys. Lett. 95, 243303 (2009)] and [Appl. Phys. Lett. 99, 123303 (2011)].

The dipole moments and LUMO energy levels according to electron buffering materials are shown in Table 3 below. Although compound BF-1 has lower barrier difference due to the LUMO energy levels of the light-emitting layer and the electron buffering layer compared to compound B-77, compound B-77 showed higher efficiency than compound BF-1. This is related to dipole moments. Compound BF-1 has a relatively large dihedral angle which results in high dipole moment value, while compound B-77 has a lower dipole moment value by having planar arrangement. Hence, compound B-77 showed fast electron current characteristics to provide high efficiency.

TABLE 3

| | Transition | |
|---|---|---|
| Electron buffering | dipole moment | LUMO |
| layer | (debye) | (eV) |
| BF-1 | 3.960 | 1.85 |
| B-77 | 0.955 | 1.92 |

In addition, from FIG. 3, it is recognized that the organic electroluminescent device of Device Example 1 showed higher current efficiency over the whole range of luminance than the organic electroluminescent device of Comparative Example 1.

TABLE 4
Compounds used in the Comparative Examples and the Device Examples

| | |
|---|---|
| Hole injection layer/ Hole transport layer | 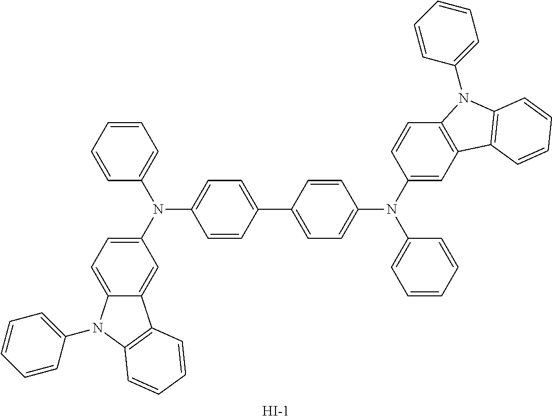 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; [I-]] |
| | 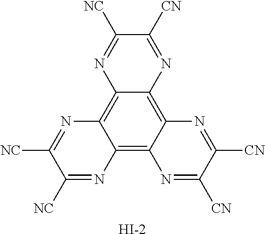 |
| | 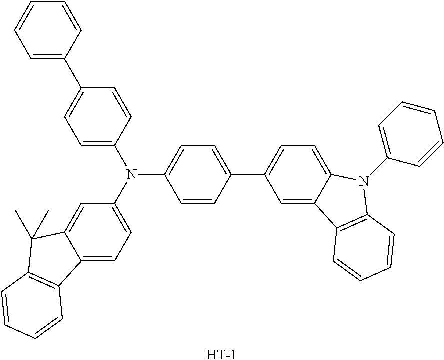 |
| | 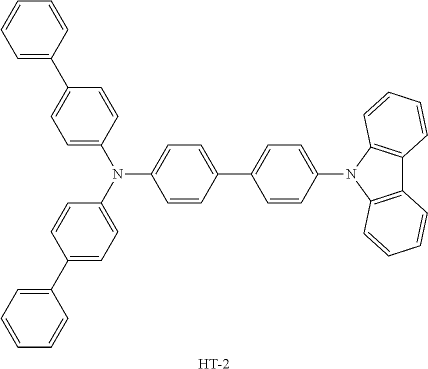 |
| Light- emitting layer | 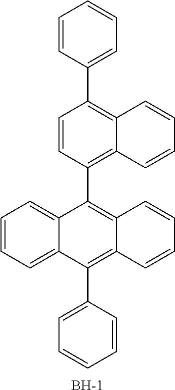 |
| | 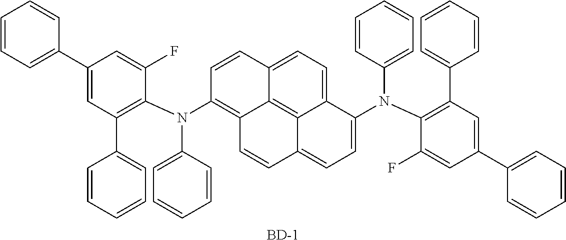 [SMILES: Fc1cc(-c2ccccc2)cc(-c2ccccc2)c1N(c1ccccc1)c1ccc2ccc3c(N(c4ccccc4)c4c(F)cc(-c5ccccc5)cc4-c4ccccc4)ccc4ccc1c2c43 ; "BD-1"] |
| Electron buffering layer | 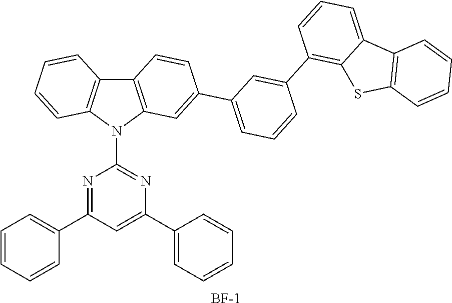 |
| Electron transport layer/ Electron injection layer | 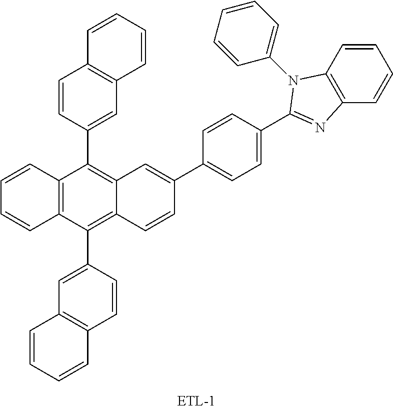 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1] |
| | 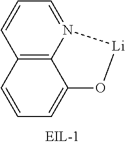 |

### REFERENCE NUMBERS

| | |
|---|---|
| 100: Organic electroluminescent device | 101: Substrate |
| 110: First electrode | 120: Organic layer |
| 122: Hole injection layer | 123: Hole transport layer |
| 125: Light-emitting layer | 126: Electron buffering layer |
| 127: Electron transport layer | 128: Electron injection layer |
| 129: Electron transport zone | 130: Second electrode |

1. An electron buffering material comprising a compound represented by the following formula 1: 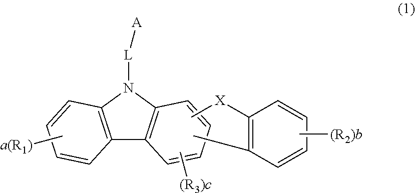
wherein
X represents O or S;
L represents a single bond, a substituted or unsubstituted (C6-C30)arylene, or a substituted or unsubstituted 5- to 30-membered heteroarylene;
A represents a substituted or unsubstituted 5- to 30-membered heteroaryl;
R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted 5- to 30-membered heteroaryl, a substituted or unsubstituted (C6-C30)aryl(C1-C30)alkyl, a substituted or unsubstituted (C3-C30)cycloalkyl, a substituted or unsubstituted (C1-C30)alkoxy, a substituted or unsubstituted (C1-C30)alkylsilyl, a substituted or unsubstituted (C6-C30)arylsilyl, a substituted or unsubstituted (C6-C30)aryl(C1-C30)alkylsilyl, a substituted or unsubstituted (C1-C30)alkylamino, a substituted or unsubstituted (C6-C30)arylamino, or a substituted or unsubstituted (C1-C30)alkyl(C6-C30)arylamino; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur;
R₃ represents hydrogen, deuterium, a halogen, a cyano, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur;
a and b each independently represent an integer of 1 to 4; where a or b is an integer of 2 or more, each of R₁ and each of R₂ may be the same or different;
c represents an integer of 1 to 2; where c is 2, each of R₃ may be the same or different; and
the heteroaryl(ene) contains at least one hetero atom selected from B, N, O, S, Si, and P.

2. The electron buffering material according to claim 1, wherein formula 1 is represented by one of the following formulae 2 to 7: 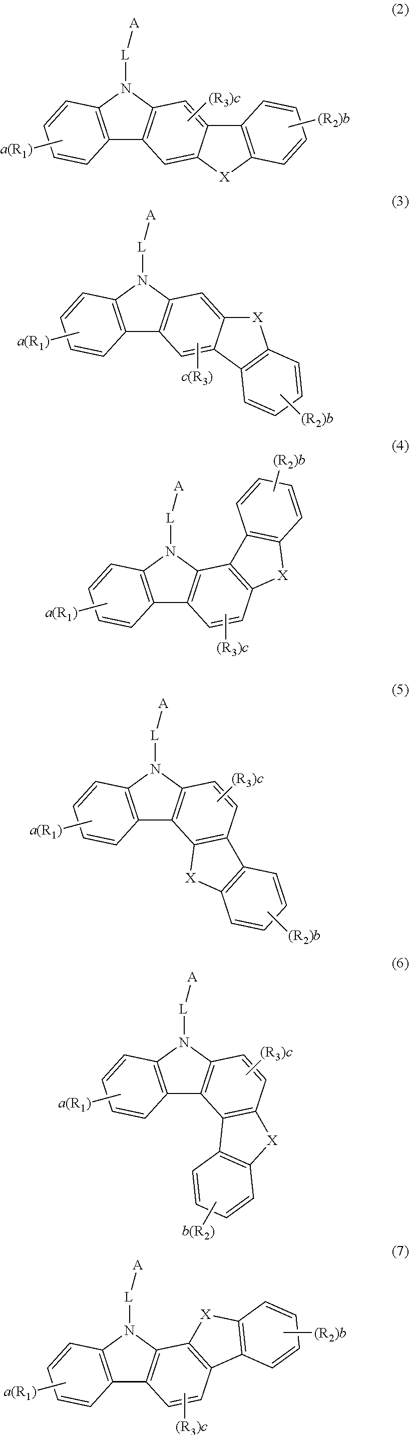
wherein
X, A, L, R₁ to R₃, a, b, and c are as defined in claim 1.

3. The electron buffering material according to claim 1, wherein the substituents of the substituted alkyl, the substituted alkoxy, the substituted cycloalkyl, the substituted aryl(ene), the substituted heteroaryl(ene), the substituted alkylsilyl, the substituted arylsilyl, the substituted arylalkylsilyl, the substituted arylamino, the substituted alkylamino, the substituted alkylarylamino, and the substituted arylalkyl in L, A, and R₁ to R₃ each independently are at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 3- to 30-membered heteroaryl unsubstituted or substituted with a (C6-C30)aryl, a (C6-C30)aryl, a (C6-C30)aryl substituted with a 3- to 30-membered heteroaryl, a (C6-C30)aryl substituted with a tri(C1-C30)alkylsilyl, a (C6-C30)aryl substituted with a tri(C6-C30)arylsilyl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

4. The electron buffering material according to claim 1, wherein
X represents O or S;
L represents a single bond, a substituted or unsubstituted (C6-C20)arylene, or a substituted or unsubstituted 5- to 20-membered heteroarylene;
A represents a substituted or unsubstituted 5- to 25-membered heteroaryl;
R₁ and R₂ each independently represent hydrogen, a substituted or unsubstituted (C6-C20)aryl, or a substituted or unsubstituted 5- to 20-membered heteroaryl;
R₃ represents hydrogen;
a and b each independently represent an integer of 1 to 2; and
c represents 1.

5. The electron buffering material according to claim 1, wherein
X represents O or S;
L represents a single bond, an unsubstituted (C6-C20)arylene, or an unsubstituted 5- to 20-membered heteroarylene;
A represents an unsubstituted 5- to 25-membered heteroaryl, a 5- to 25-membered heteroaryl substituted with a cyano, a 5- to 25-membered heteroaryl substituted with a (C6-C25)aryl, a 5- to 25-membered heteroaryl substituted with a 5- to 20-membered heteroaryl, or a 5- to 25-membered heteroaryl substituted with a (C1-C6)alkyl(C6-C20)aryl;
R₁ and R₂ each independently represent hydrogen, a (C6-C20)aryl unsubstituted or substituted with a (C1-C6)alkyl, or a 5- to 20-membered heteroaryl unsubstituted or substituted with a (C6-C20)aryl;
R₃ represents hydrogen;
a and b each independently represent an integer of 1 to 2; and
c represents 1.

6. The electron buffering material according to claim 1, wherein A represents a substituted or unsubstituted pyridine, a substituted or unsubstituted pyrimidine, a substituted or unsubstituted triazine, a substituted or unsubstituted pyrazine, a substituted or unsubstituted quinoline, a substituted or unsubstituted quinazoline, a substituted or unsubstituted quinoxaline, a substituted or unsubstituted benzimidazole, a substituted or unsubstituted naphthyridine, or a substituted or unsubstituted phenanthroline.

7. The electron buffering material according to claim 1, wherein the compound represented by formula 1 is selected from the group consisting of:

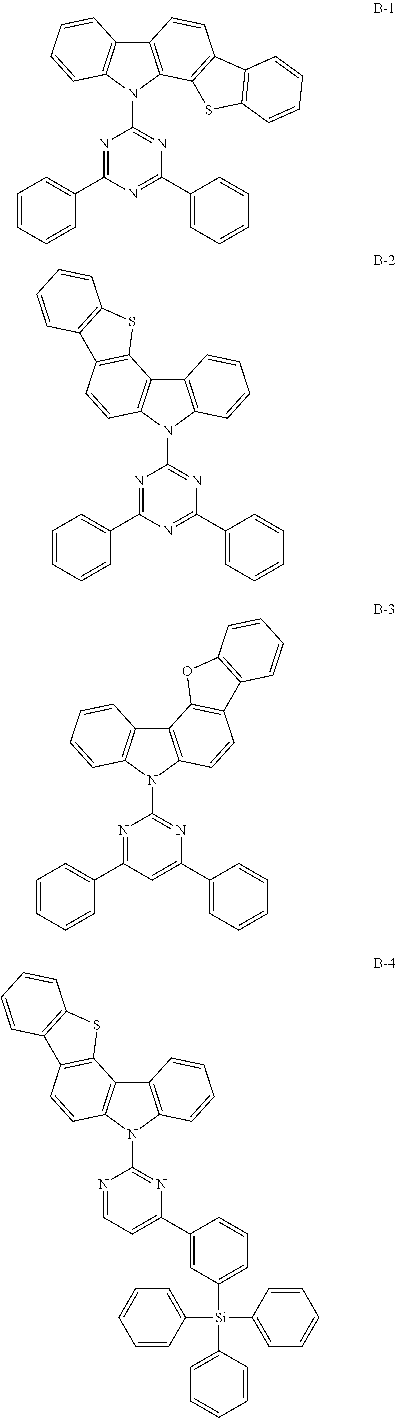

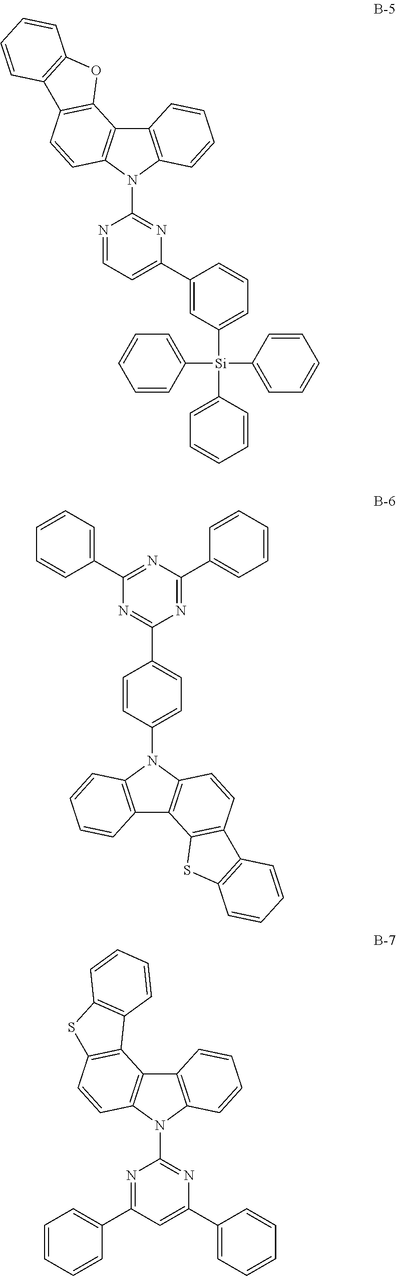 [SMILES: c1ccc([Si](c2ccccc2)(c2ccccc2)c2cccc(-c3ccnc(-n4c5ccccc5c5c6oc7ccccc7c6ccc54)n3)c2)cc1 ; c1ccc(-c2nc(-c3ccccc3)nc(-c3ccc(-n4c5ccccc5c5c6sc7ccccc7c6ccc54)cc3)n2)cc1 ; c1ccc(-c2cc(-c3ccccc3)nc(-n3c4ccccc4c4c5c(ccc43)sc3ccccc35)n2)cc1]

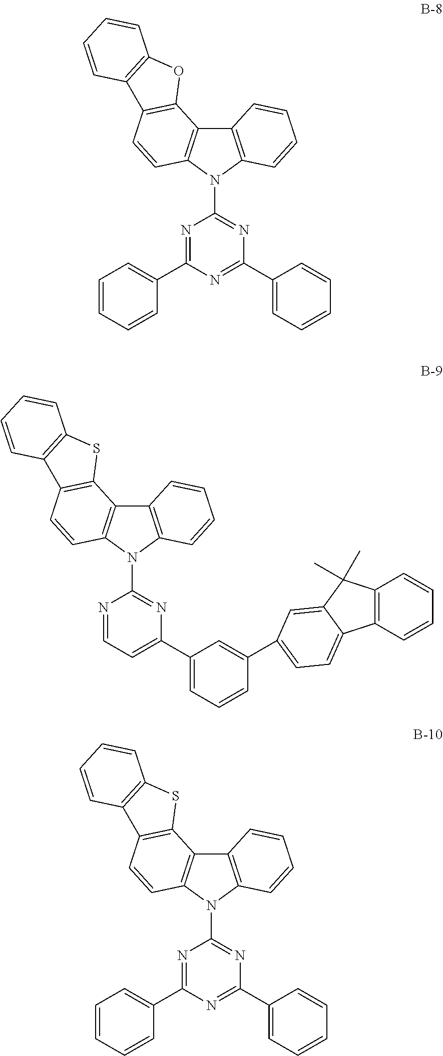 [SMILES: c1ccc(-c2nc(-c3ccccc3)nc(-n3c4ccccc4c4c5oc6ccccc6c5ccc43)n2)cc1 ; CC1(C)c2ccccc2-c2ccc(-c3cccc(-c4ccnc(-n5c6ccccc6c6c7sc8ccccc8c7ccc65)n4)c3)cc21 ; c1ccc(-c2nc(-c3ccccc3)nc(-n3c4ccccc4c4c5sc6ccccc6c5ccc43)n2)cc1]

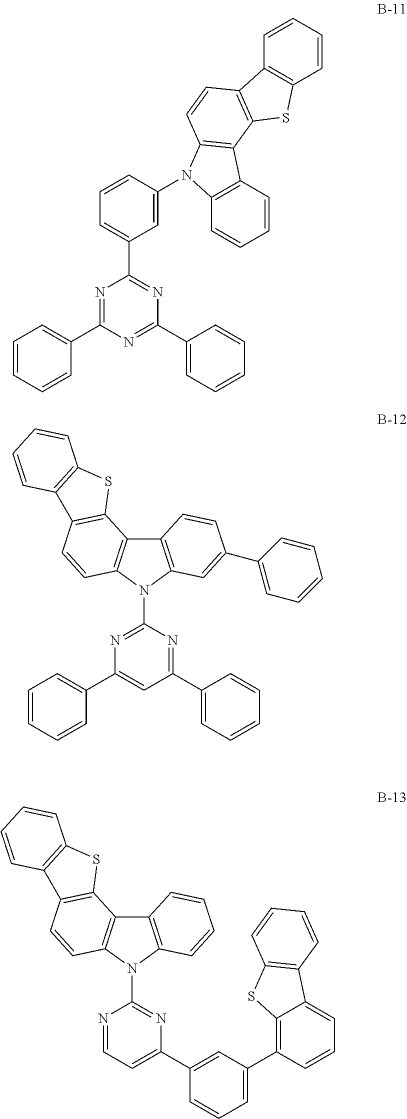

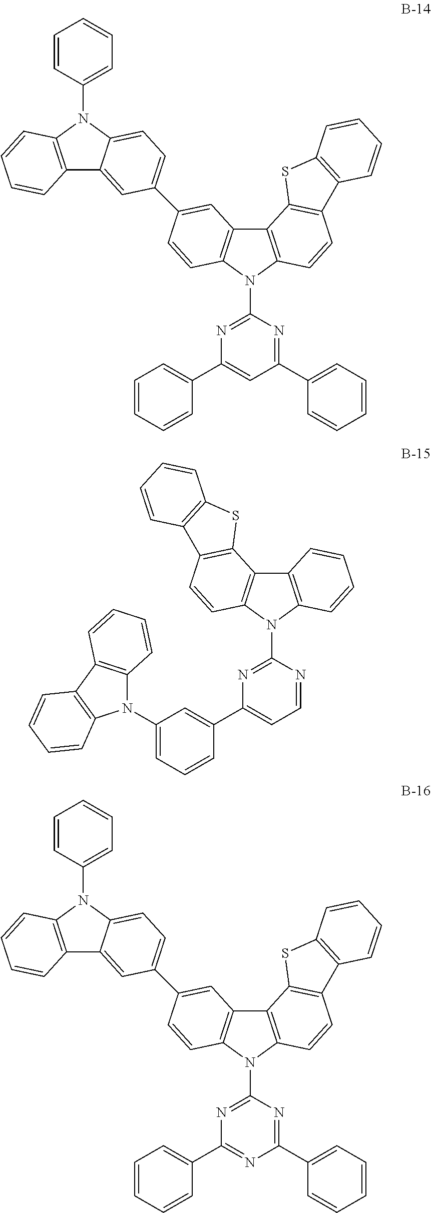

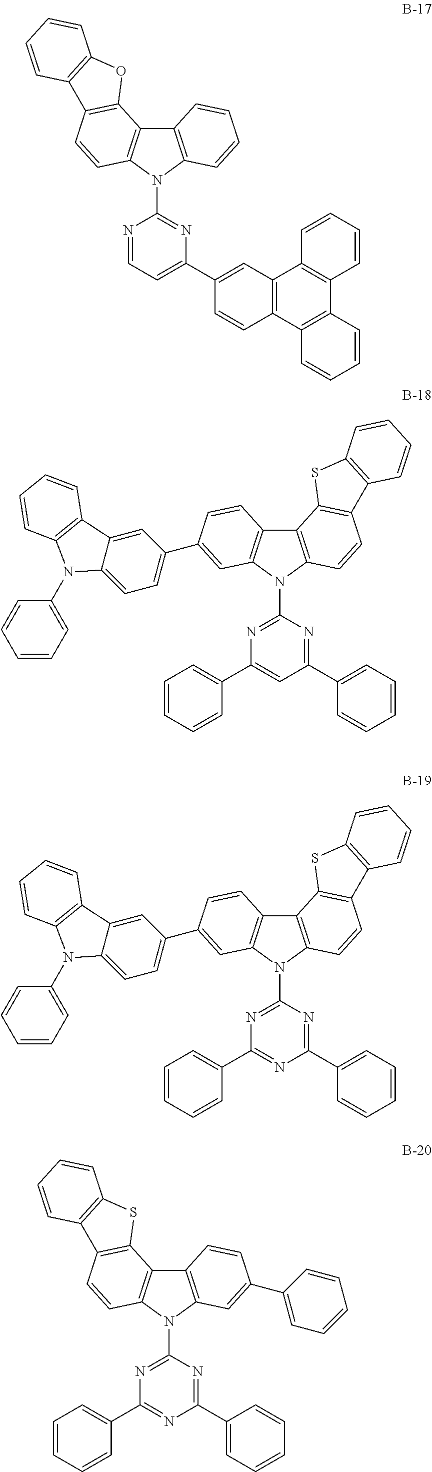 [SMILES: c1ccc2c(c1)oc1c2ccc2c1c1ccccc1n2-c1nccc(-c2ccc3c4ccccc4c4ccccc4c3c2)n1 ; c1ccc(-c2cc(-c3ccccc3)nc(-n3c4cc(-c5ccc6c(c5)c5ccccc5n6-c5ccccc5)ccc4c4c5sc6ccccc6c5ccc43)n2)cc1 ; c1ccc(-c2nc(-c3ccccc3)nc(-n3c4cc(-c5ccc6c(c5)c5ccccc5n6-c5ccccc5)ccc4c4c5sc6ccccc6c5ccc43)n2)cc1 ; c1ccc(-c2ccc3c4c5sc6ccccc6c5ccc4n(-c4nc(-c5ccccc5)nc(-c5ccccc5)n4)c3c2)cc1]

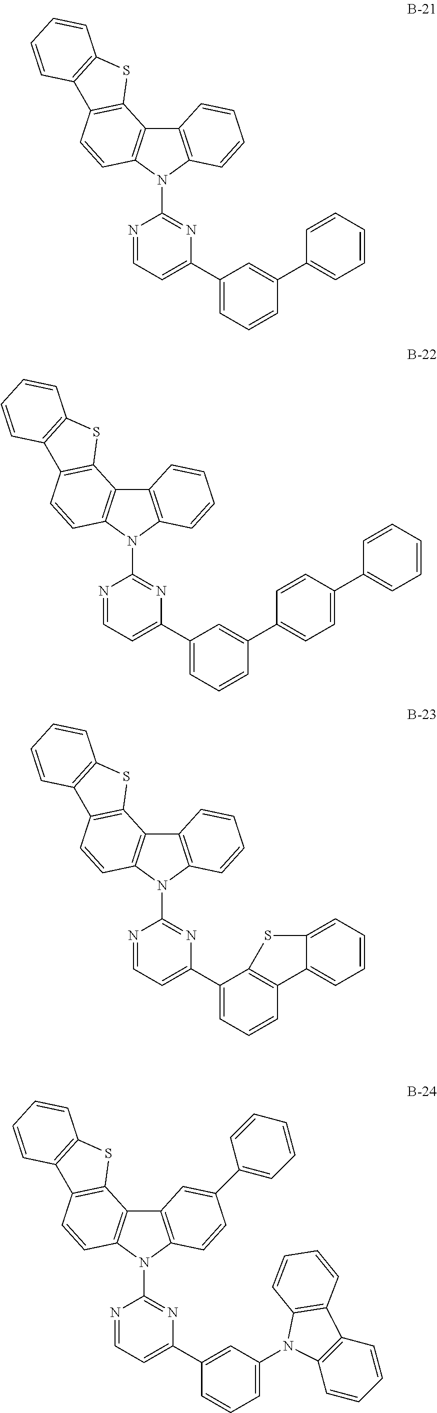 [SMILES: c1ccc(-c2cccc(-c3ccnc(-n4c5ccccc5c5c6sc7ccccc7c6ccc54)n3)c2)cc1 ; c1ccc(-c2ccc(-c3cccc(-c4ccnc(-n5c6ccccc6c6c7sc8ccccc8c7ccc65)n4)c3)cc2)cc1 ; c1ccc2c(c1)sc1c(-c3ccnc(-n4c5ccccc5c5c6sc7ccccc7c6ccc54)n3)cccc12 ; c1ccc(-c2ccc3c(c2)c2c4sc5ccccc5c4ccc2n3-c2nccc(-c3cccc(-n4c5ccccc5c5ccccc54)c3)n2)cc1]

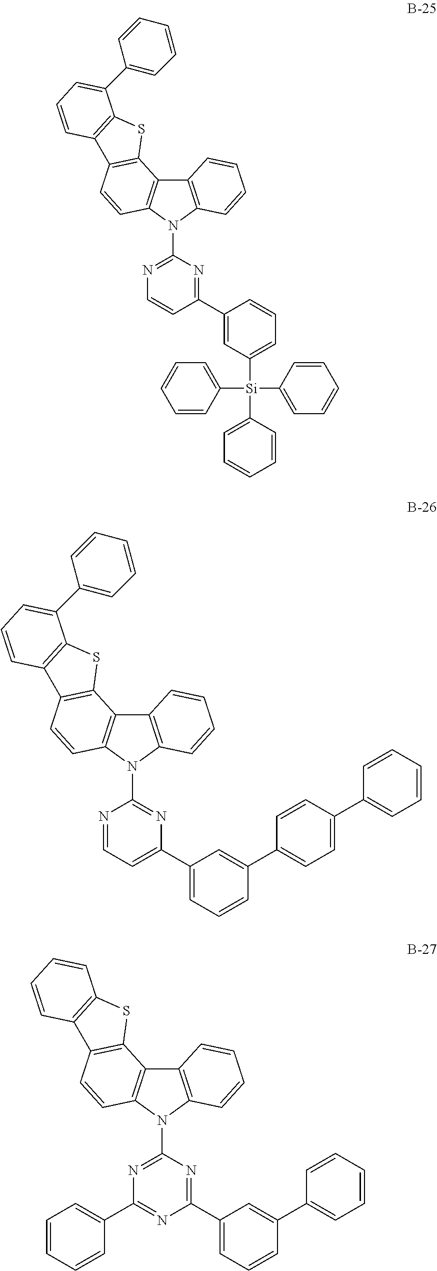 [SMILES: c1ccc(-c2cccc3c2sc2c3ccc3c2c2ccccc2n3-c2nccc(-c3cccc([Si](c4ccccc4)(c4ccccc4)c4ccccc4)c3)n2)cc1 ; c1ccc(-c2ccc(-c3cccc(-c4ccnc(-n5c6ccccc6c6c7sc8c(-c9ccccc9)cccc8c7ccc65)n4)c3)cc2)cc1 ; c1ccc(-c2cccc(-c3nc(-c4ccccc4)nc(-n4c5ccccc5c5c6sc7ccccc7c6ccc54)n3)c2)cc1]

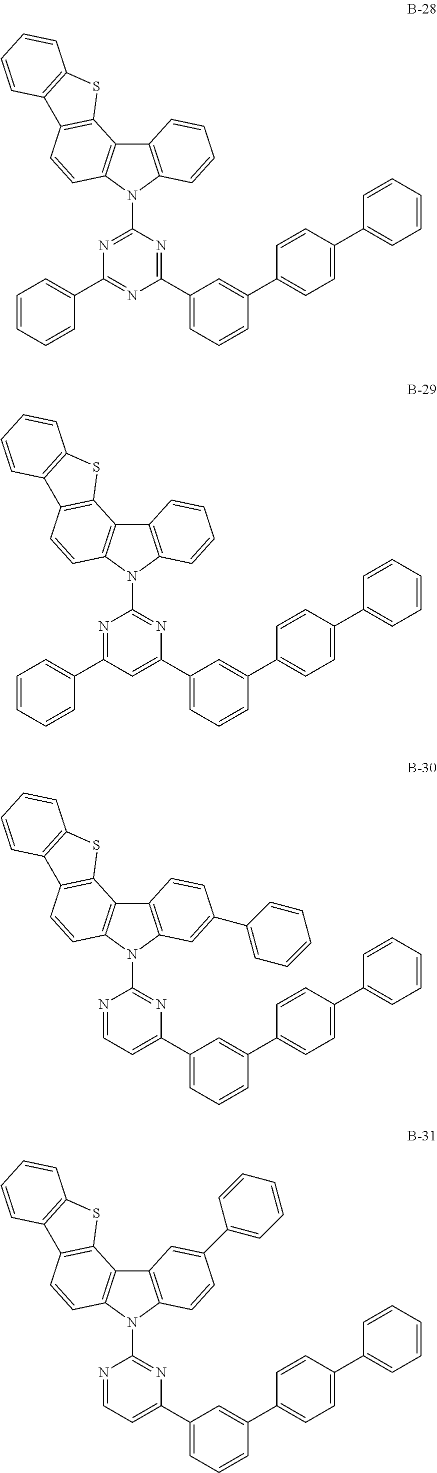 [SMILES: c1ccc(-c2ccc(-c3cccc(-c4nc(-c5ccccc5)nc(-n5c6ccccc6c6c7sc8ccccc8c7ccc65)n4)c3)cc2)cc1 ; c1ccc(-c2ccc(-c3cccc(-c4cc(-c5ccccc5)nc(-n5c6ccccc6c6c7sc8ccccc8c7ccc65)n4)c3)cc2)cc1 ; c1ccc(-c2ccc(-c3cccc(-c4ccnc(-n5c6cc(-c7ccccc7)ccc6c6c7sc8ccccc8c7ccc65)n4)c3)cc2)cc1 ; c1ccc(-c2ccc(-c3cccc(-c4ccnc(-n5c6ccc(-c7ccccc7)cc6c6c7sc8ccccc8c7ccc65)n4)c3)cc2)cc1]

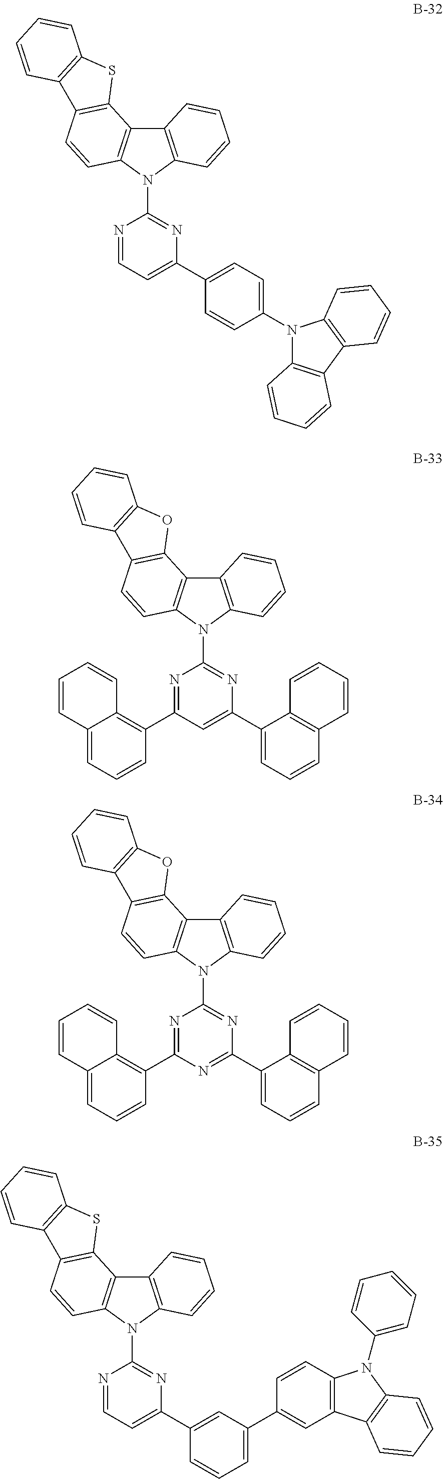

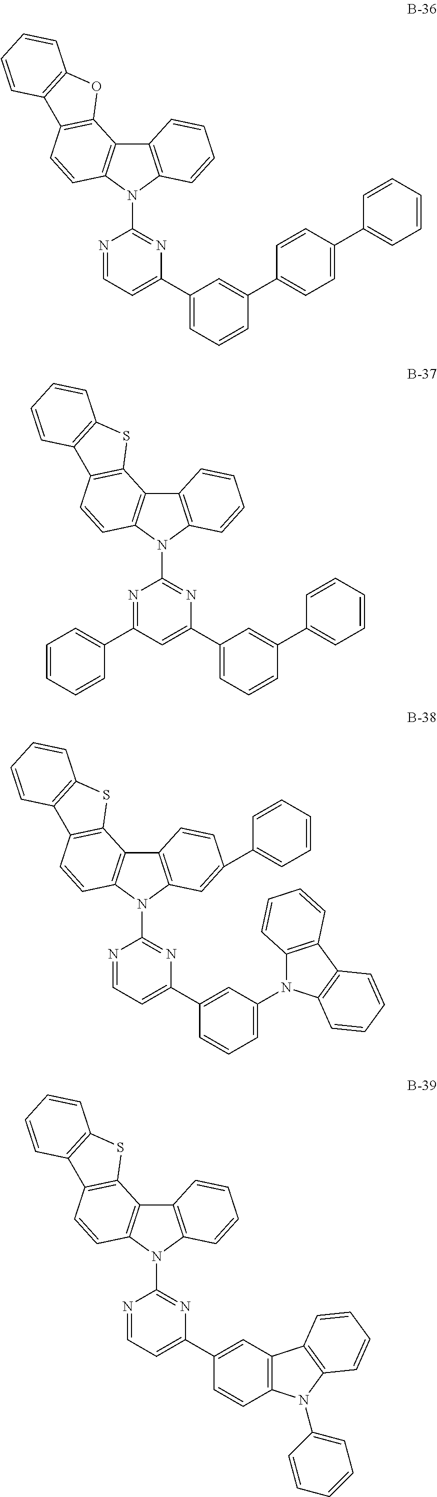 [SMILES: c1ccc(-c2ccc(-c3cccc(-c4ccnc(-n5c6ccccc6c6c7oc8ccccc8c7ccc65)n4)c3)cc2)cc1 ; c1ccc(-c2cccc(-c3cc(-c4ccccc4)nc(-n4c5ccccc5c5c6sc7ccccc7c6ccc54)n3)c2)cc1 ; c1ccc(-c2ccc3c4c5sc6ccccc6c5ccc4n(-c4nccc(-c5cccc(-n6c7ccccc7c7ccccc76)c5)n4)c3c2)cc1 ; c1ccc(-n2c3ccccc3c3cc(-c4ccnc(-n5c6ccccc6c6c7sc8ccccc8c7ccc65)n4)ccc32)cc1]

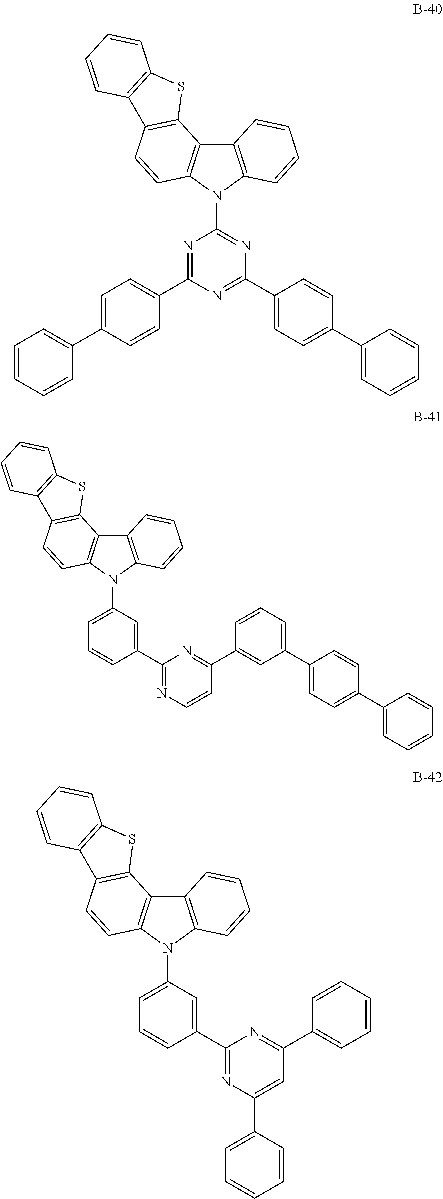 [SMILES: c1ccc(-c2ccc(-c3nc(-c4ccc(-c5ccccc5)cc4)nc(-n4c5ccccc5c5c6sc7ccccc7c6ccc54)n3)cc2)cc1 ; c1ccc(-c2ccc(-c3cccc(-c4ccnc(-c5cccc(-n6c7ccccc7c7c8sc9ccccc9c8ccc76)c5)n4)c3)cc2)cc1 ; c1ccc(-c2cc(-c3ccccc3)nc(-c3cccc(-n4c5ccccc5c5c6sc7ccccc7c6ccc54)c3)n2)cc1]

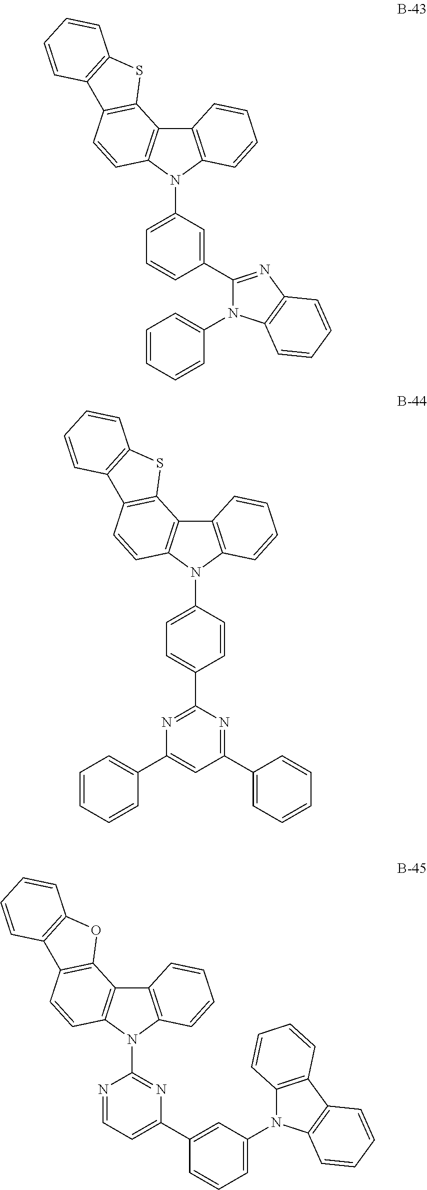 [SMILES: c1ccc(-n2c(-c3cccc(-n4c5ccccc5c5c6sc7ccccc7c6ccc54)c3)nc3ccccc32)cc1 ; c1ccc(-c2cc(-c3ccccc3)nc(-c3ccc(-n4c5ccccc5c5c6sc7ccccc7c6ccc54)cc3)n2)cc1 ; c1cc(-c2ccnc(-n3c4ccccc4c4c5oc6ccccc6c5ccc43)n2)cc(-n2c3ccccc3c3ccccc32)c1]

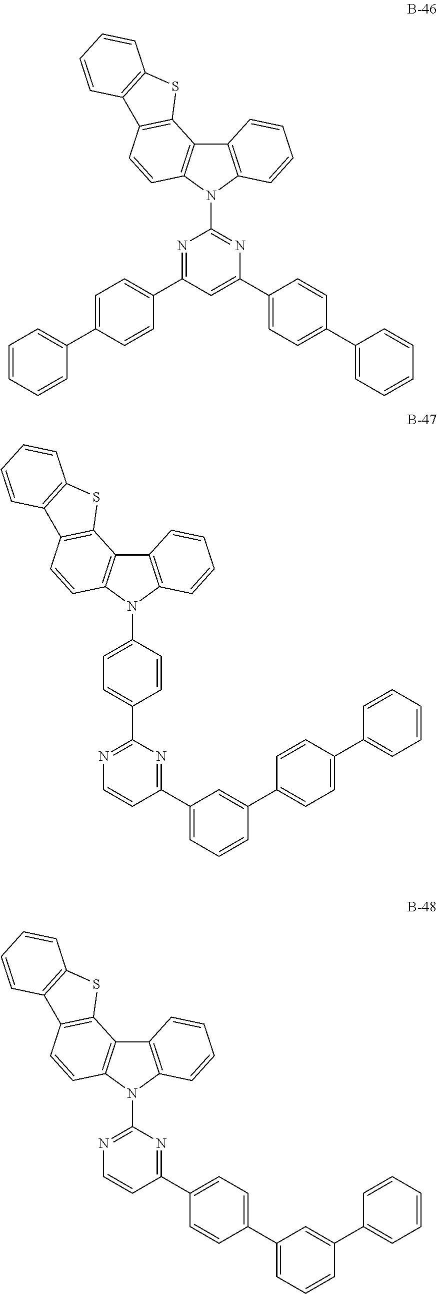 [SMILES: c1ccc(-c2ccc(-c3cc(-c4ccc(-c5ccccc5)cc4)nc(-n4c5ccccc5c5c6sc7ccccc7c6ccc54)n3)cc2)cc1 ; c1ccc(-c2ccc(-c3cccc(-c4ccnc(-c5ccc(-n6c7ccccc7c7c8sc9ccccc9c8ccc76)cc5)n4)c3)cc2)cc1 ; c1ccc(-c2cccc(-c3ccc(-c4ccnc(-n5c6ccccc6c6c7sc8ccccc8c7ccc65)n4)cc3)c2)cc1]

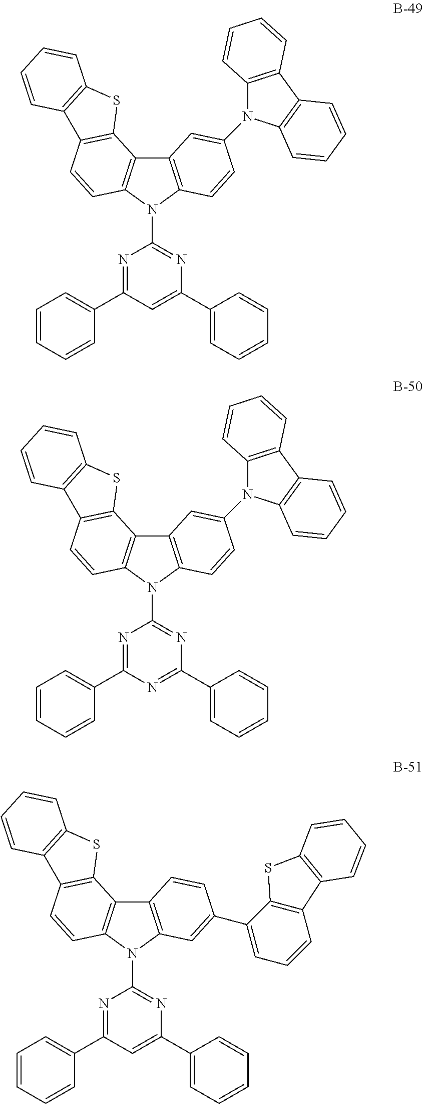

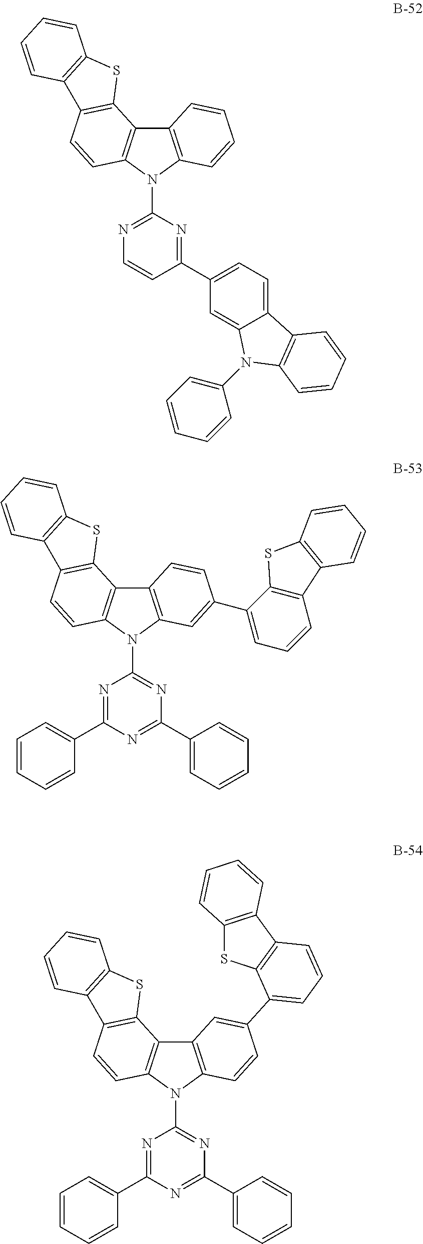 [SMILES: c1ccc(-n2c3ccccc3c3ccc(-c4ccnc(-n5c6ccccc6c6c7sc8ccccc8c7ccc65)n4)cc32)cc1 ; c1ccc(-c2nc(-c3ccccc3)nc(-n3c4cc(-c5cccc6c5sc5ccccc56)ccc4c4c5sc6ccccc6c5ccc43)n2)cc1 ; c1ccc(-c2nc(-c3ccccc3)nc(-n3c4ccc(-c5cccc6c5sc5ccccc56)cc4c4c5sc6ccccc6c5ccc43)n2)cc1]

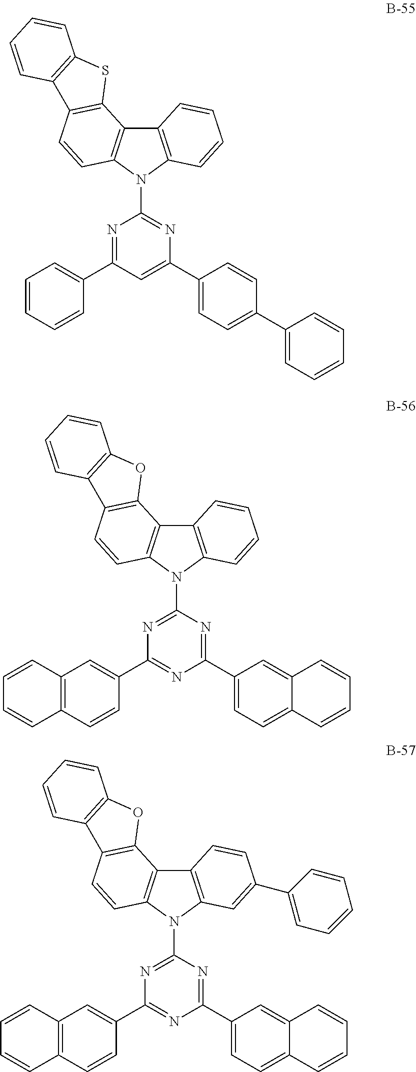

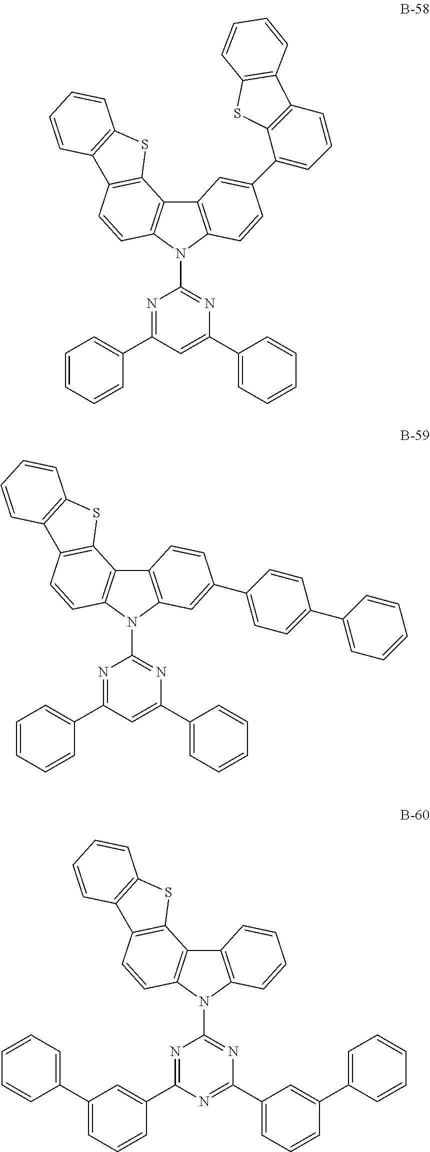 [SMILES: c1ccc(-c2cc(-c3ccccc3)nc(-n3c4ccc(-c5cccc6c5sc5ccccc56)cc4c4c5sc6ccccc6c5ccc43)n2)cc1 ; c1ccc(-c2ccc(-c3ccc4c5c6sc7ccccc7c6ccc5n(-c5nc(-c6ccccc6)cc(-c6ccccc6)n5)c4c3)cc2)cc1 ; c1ccc(-c2cccc(-c3nc(-c4cccc(-c5ccccc5)c4)nc(-n4c5ccccc5c5c6sc7ccccc7c6ccc54)n3)c2)cc1]

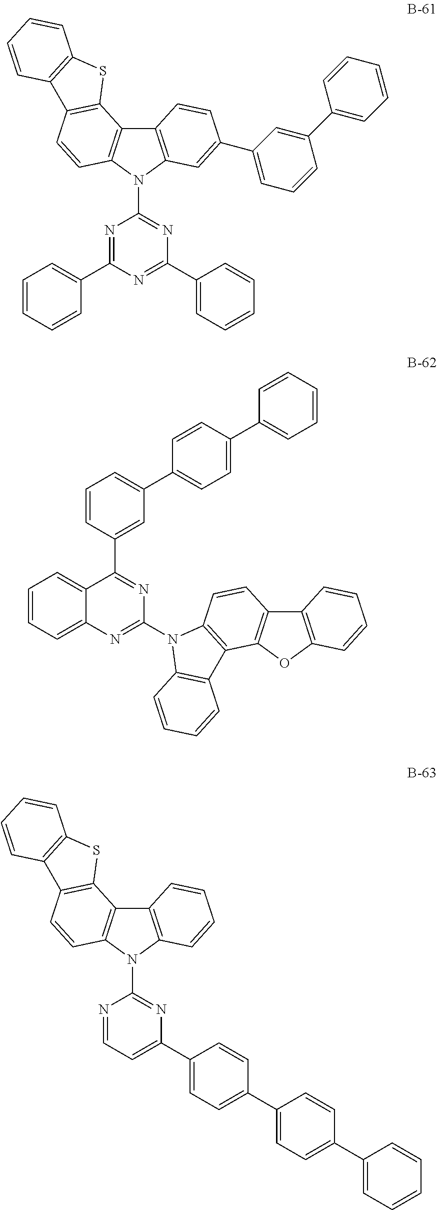 [SMILES: c1ccc(-c2cccc(-c3ccc4c5c6sc7ccccc7c6ccc5n(-c5nc(-c6ccccc6)nc(-c6ccccc6)n5)c4c3)c2)cc1 ; c1ccc(-c2ccc(-c3cccc(-c4nc(-n5c6ccccc6c6c7oc8ccccc8c7ccc65)nc5ccccc45)c3)cc2)cc1 ; c1ccc(-c2ccc(-c3ccc(-c4ccnc(-n5c6ccccc6c6c7sc8ccccc8c7ccc65)n4)cc3)cc2)cc1]

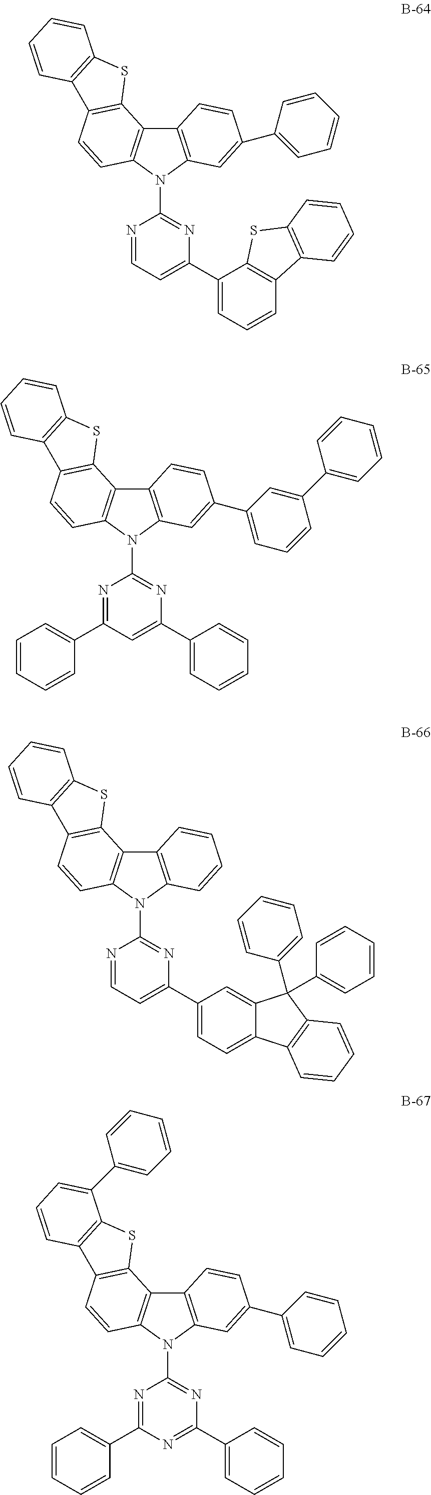 [SMILES: c1ccc(-c2ccc3c4c5sc6ccccc6c5ccc4n(-c4nccc(-c5cccc6c5sc5ccccc56)n4)c3c2)cc1 ; c1ccc(-c2cccc(-c3ccc4c5c6sc7ccccc7c6ccc5n(-c5nc(-c6ccccc6)cc(-c6ccccc6)n5)c4c3)c2)cc1 ; c1ccc(C2(c3ccccc3)c3ccccc3-c3ccc(-c4ccnc(-n5c6ccccc6c6c7sc8ccccc8c7ccc65)n4)cc32)cc1 ; c1ccc(-c2ccc3c4c5sc6c(-c7ccccc7)cccc6c5ccc4n(-c4nc(-c5ccccc5)nc(-c5ccccc5)n4)c3c2)cc1]

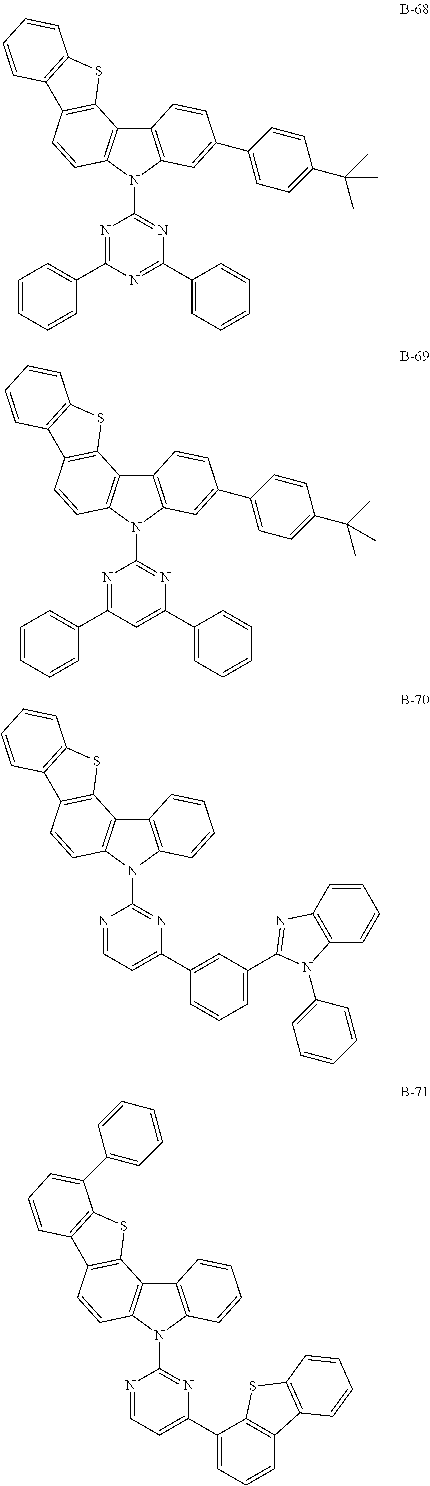

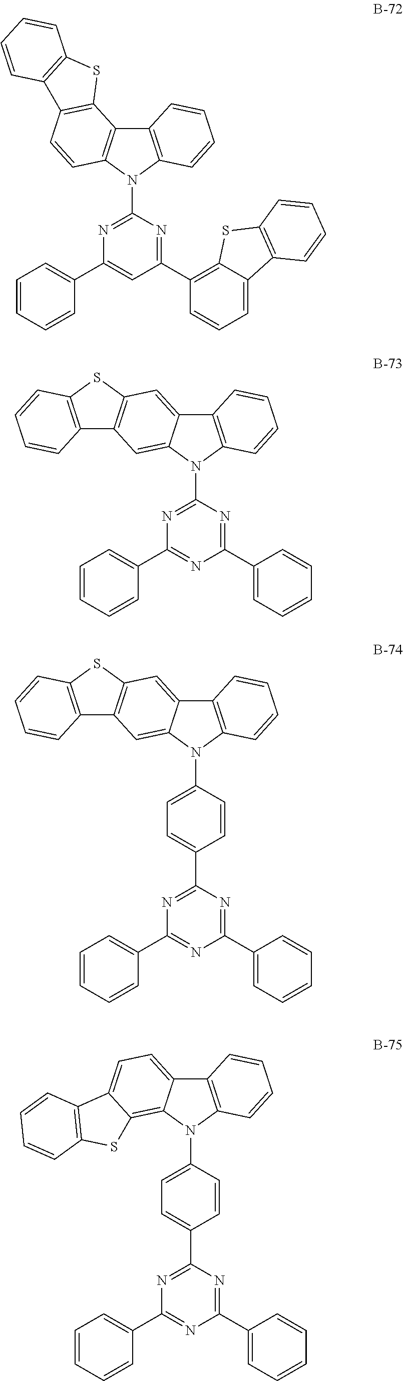 [SMILES: c1ccc(-c2cc(-c3cccc4c3sc3ccccc34)nc(-n3c4ccccc4c4c5sc6ccccc6c5ccc43)n2)cc1 ; c1ccc(-c2nc(-c3ccccc3)nc(-n3c4ccccc4c4cc5sc6ccccc6c5cc43)n2)cc1 ; c1ccc(-c2nc(-c3ccccc3)nc(-c3ccc(-n4c5ccccc5c5cc6sc7ccccc7c6cc54)cc3)n2)cc1 ; c1ccc(-c2nc(-c3ccccc3)nc(-c3ccc(-n4c5ccccc5c5ccc6c7ccccc7sc6c54)cc3)n2)cc1]

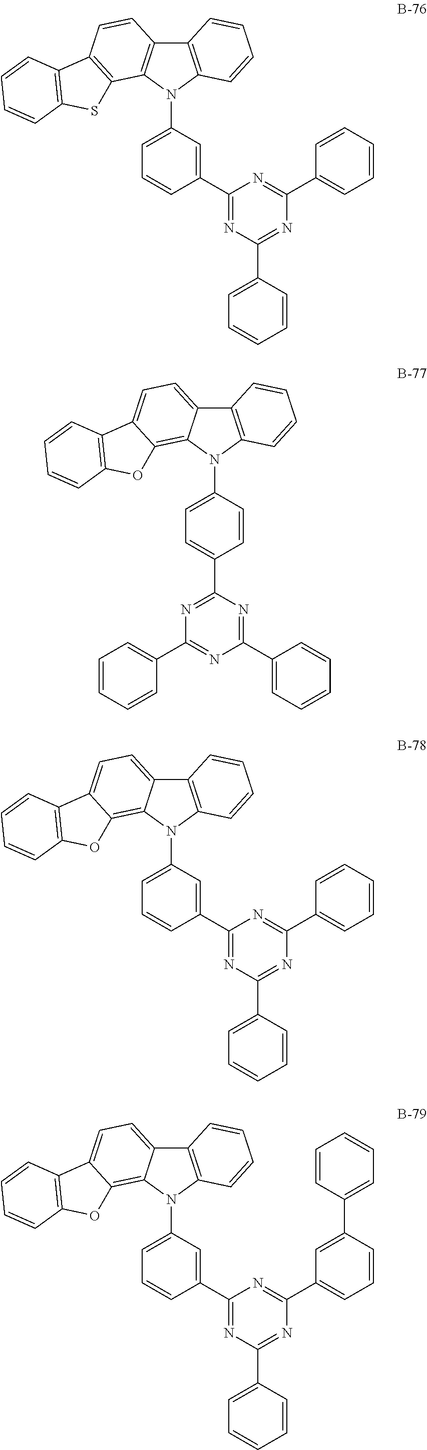

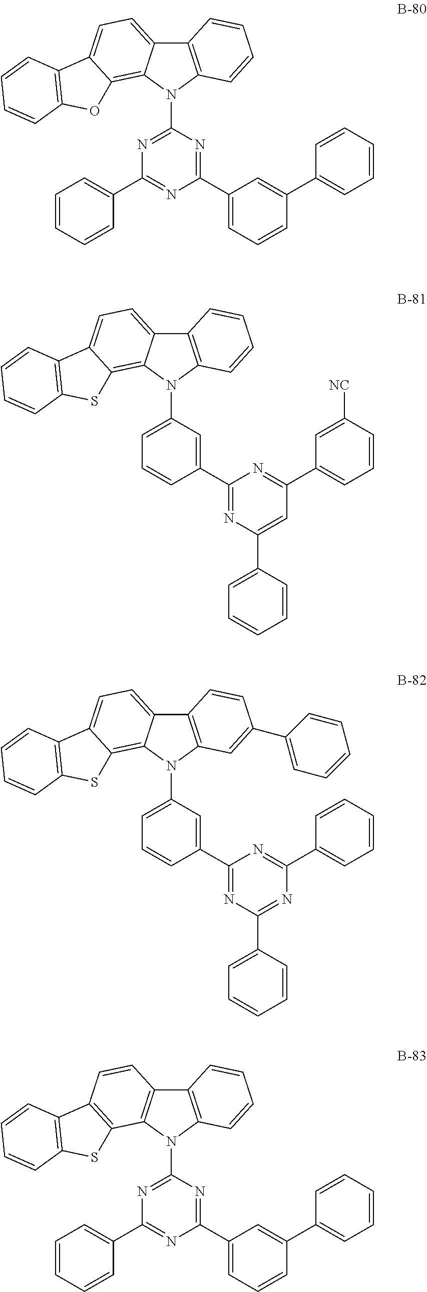 [SMILES: c1ccc(-c2cccc(-c3nc(-c4ccccc4)nc(-n4c5ccccc5c5ccc6c7ccccc7oc6c54)n3)c2)cc1 ; N#Cc1cccc(-c2cc(-c3ccccc3)nc(-c3cccc(-n4c5ccccc5c5ccc6c7ccccc7sc6c54)c3)n2)c1 ; c1ccc(-c2ccc3c4ccc5c6ccccc6sc5c4n(-c4cccc(-c5nc(-c6ccccc6)nc(-c6ccccc6)n5)c4)c3c2)cc1 ; c1ccc(-c2cccc(-c3nc(-c4ccccc4)nc(-n4c5ccccc5c5ccc6c7ccccc7sc6c54)n3)c2)cc1]

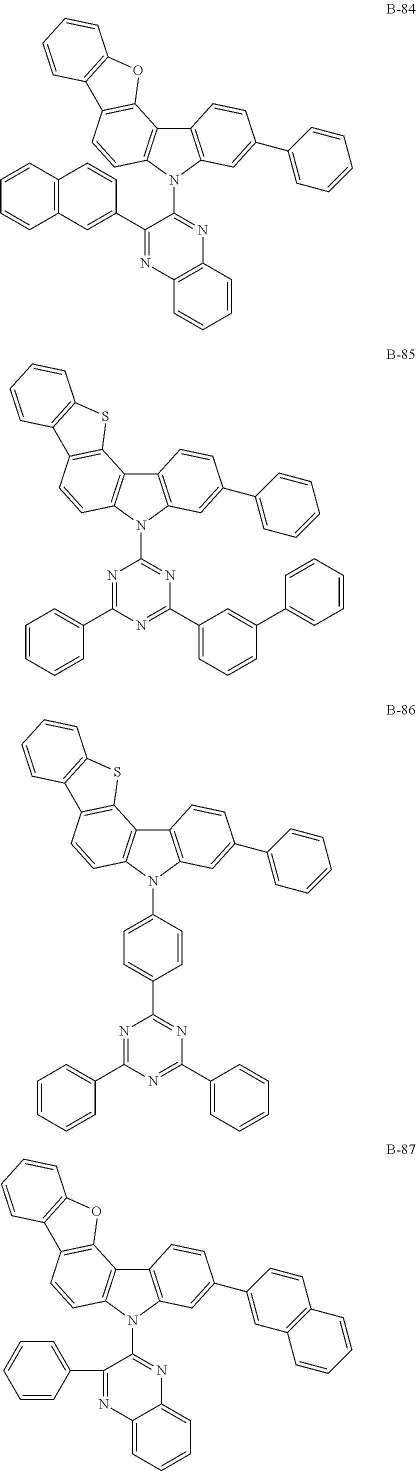

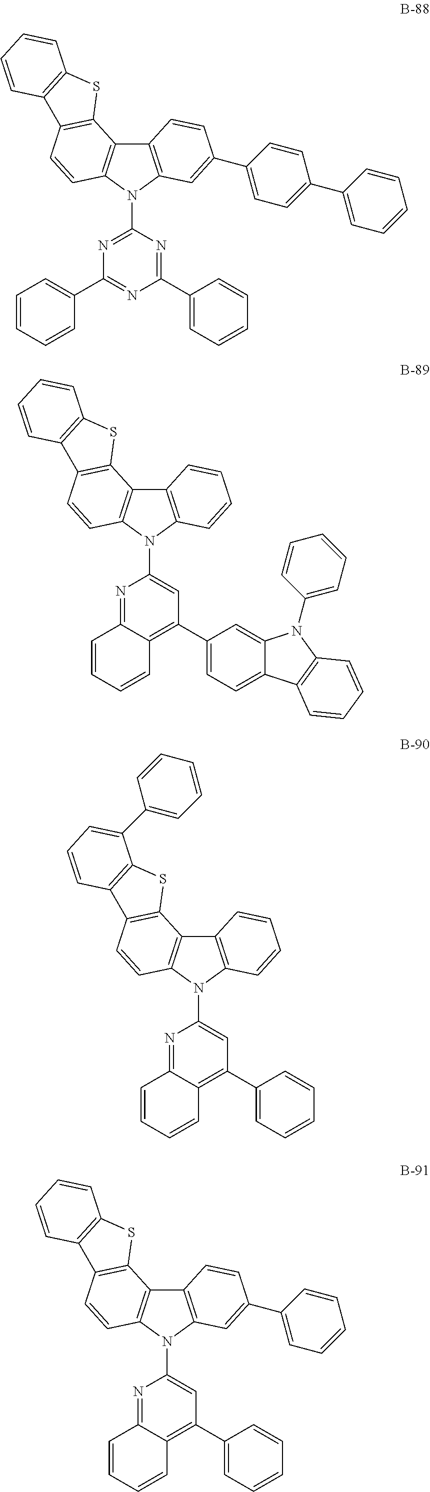

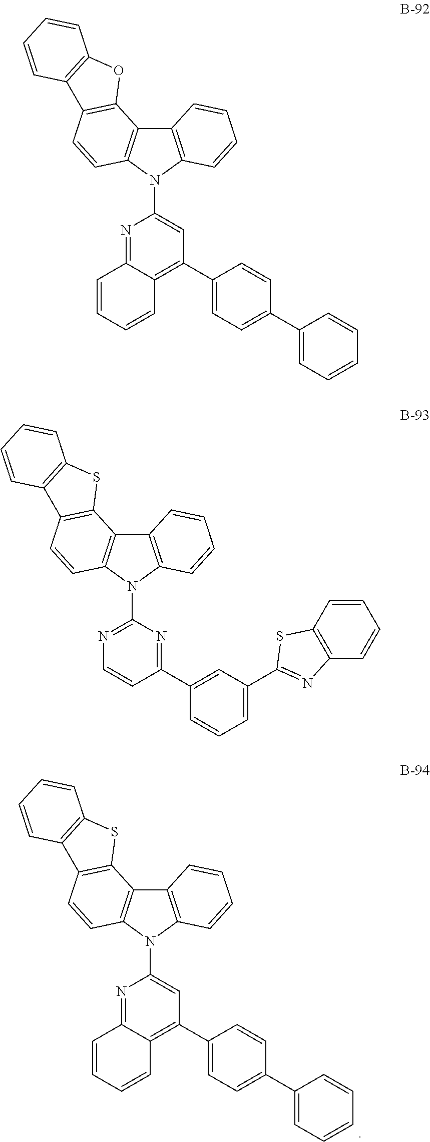 [SMILES: c1ccc(-c2ccc(-c3cc(-n4c5ccccc5c5c6oc7ccccc7c6ccc54)nc4ccccc34)cc2)cc1 ; c1cc(-c2ccnc(-n3c4ccccc4c4c5sc6ccccc6c5ccc43)n2)cc(-c2nc3ccccc3s2)c1 ; c1ccc(-c2ccc(-c3cc(-n4c5ccccc5c5c6sc7ccccc7c6ccc54)nc4ccccc34)cc2)cc1]

8. An organic electroluminescent device comprising a first electrode; a second electrode facing the first electrode; a light-emitting layer between the first electrode and the second electrode; and an electron transport zone and an electron buffering layer between the light-emitting layer and the second electrode;
wherein the electron buffering layer comprises a compound represented by the following formula 1: 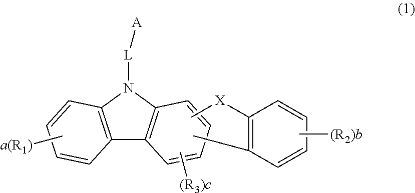
wherein
X represents O or S;
L represents a single bond, a substituted or unsubstituted (C6-C30)arylene, or a substituted or unsubstituted 5- to 30-membered heteroarylene;
A represents a substituted or unsubstituted 5- to 30-membered heteroaryl;
R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted 5- to 30-membered heteroaryl, a substituted or unsubstituted (C6-C30)aryl(C1-C30)alkyl, a substituted or unsubstituted (C3-C30)cycloalkyl, a substituted or unsubstituted (C1-C30)alkoxy, a substituted or unsubstituted (C1-C30)alkylsilyl, a substituted or unsubstituted (C6-C30)arylsilyl, a substituted or unsubstituted (C6-C30)aryl(C1-C30)alkylsilyl, a substituted or unsubstituted (C1-C30)alkylamino, a substituted or unsubstituted (C6-C30)arylamino, or a substituted or unsubstituted (C1-C30)alkyl(C6-C30)arylamino; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur;
R₃ represents hydrogen, deuterium, a halogen, a cyano, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur;
a and b each independently represent an integer of 1 to 4; where a or b is an integer of 2 or more, each of R₁ and each of R₂ may be the same or different;
c represents an integer of 1 to 2; where c is 2, each of R₃ may be the same or different; and
the heteroaryl(ene) contains at least one hetero atom selected from B, N, O, S, Si, and P.

9. The organic electroluminescent device according to claim 8, wherein the light-emitting layer comprises a host compound and a dopant compound, and the LUMO (lowest unoccupied molecular orbital) energy level of the electron buffering layer is greater than the LUMO energy level of the host compound.

10. The organic electroluminescent device according to claim 8, wherein the electron transport zone comprises an electron transport compound, a reductive dopant, or a combination thereof.

11. The organic electroluminescent device according to claim 10, wherein the electron transport compound is at least one selected from the group consisting of oxazole-based compounds, isoxazole-based compounds, triazole-based compounds, isothiazole-based compounds, oxadiazole-based compounds, thiadiazole-based compounds, perylene-based compounds, anthracene-based compounds, aluminum complexes, and gallium complexes; and the reductive dopant is at least one selected from the group consisting of alkali metals, alkali metal compounds, alkaline-earth metals, rare-earth metals, halides thereof, oxides thereof, and complexes thereof.

12. The organic electroluminescent device according to claim 8, wherein the electron transport zone comprises an electron injection layer, an electron transport layer, or both of them.

13. The organic electroluminescent device according to claim 8, further comprising a hole injection layer, a hole transport layer, or both of them between the first electrode and the light-emitting layer.
